(19) United States
(12) Patent Application Publication
(10) Pub. No.: US 2022/0228177 A1
(43) Pub. Date: Jul. 21, 2022

(54) RECOMBINANT MICROORGANISM HAVING HIGH ABILITY TO PRODUCE LUTEIN AND METHOD FOR PRODUCING LUTEIN USING THE SAME

(71) Applicant: KOREA ADVANCED INSTITUTE OF SCIENCE AND TECHNOLOGY, Daejeon (KR)

(72) Inventors: Sang Yup LEE, Daejeon (KR); Seon Young PARK, Daejeon (KR)

(21) Appl. No.: 17/575,460

(22) Filed: Jan. 13, 2022

(30) Foreign Application Priority Data
Jan. 15, 2021 (KR) 10-2021-0005806

(51) Int. Cl. C12P 7/22; C12N 1/20; C12N 15/11

(52) U.S. Cl. CPC C12P 7/22; C12N 15/111; C12N 1/205

(57) ABSTRACT

The present invention relates to a recombinant microorganism having enhanced ability to produce lutein and a method of producing lutein using the same, and more specifically, to a recombinant microorganism having enhanced ability to produce lutein, which is obtained by modifying any one or more metabolic pathways, selected from the group consisting of a substrate tunnel, an electron tunnel, and a C5 heme production pathway, in a host cell having ability to produce lutein. Using the highly efficient lutein-producing recombinant microbial strain according to the present invention, it is possible to replace an existing lutein production method that relies on labor-intensive and inefficient plant extraction and to produce lutein in a more environmentally friendly and sustainable way. In addition, the strain development strategy used in the present invention is useful because it may be used to construct a recombinant strain for the efficient production of useful compounds with complex metabolic pathways and to establish an efficient production method, and it may be applied throughout the gradually expanding biochemical market.

### TECHNICAL FIELD

The present invention relates to a recombinant microorganism having high ability to produce lutein and a method of producing lutein using the same, and more specifically, to a recombinant microorganism having ability to produce lutein, which is obtained by introducing a lutein biosynthetic pathway into a host cell having ability to produce farnesyl diphosphate (FPP).

### BACKGROUND ART

Lutein is one of the xanthophylls naturally found in egg yolks, fruits, and green leafy vegetables. Lutein is abundant in the macula of the human eye and functions to provide protection against oxidative stress and radiation. Consuming such lutein can prevent macular degeneration and cataracts, protect the skin from UV rays, and help prevent cancer and cardiovascular disease. Due to these effects, the demand and market for lutein are increasing.

Lutein that is supplied to the market is mainly extracted from marigold flowers, but the process of purifying lutein from the extract is complicated because marigold flowers also produce esterified lutein which is difficult to separate from lutein. Since the chemical structure of lutein is asymmetrical and various lutein isomers exist, chemical synthesis of lutein is also inefficient. Efforts have been made to overproduce lutein by manipulating the genes of plants and microalgae, but the lutein production and productivity have not met expectations.

For example, U.S. Pat. No. 5,530,189 describes a recombinant plant obtained by introducing crtE, crtB and crtl genes into a plant and producing an increased amount of lutein, but does not report on a recombinant microorganism having ability to produce lutein. U.S. Pat. No. 10,059,974 describes a recombinant microorganism having ability to produce lutein, which is obtained by introducing genes encoding CYP97A, CYP97B and CYP97C proteins and genes encoding lycopene cyclase proteins, but has a disadvantage in that lutein is produced at a rate similar to or lower than beta-carotene.

Korean Patent No. 10-1339686 describes a method for producing chlorella with a high lutein content, but this method is a method of increasing the content by modifying the culture conditions rather than modifying the metabolic pathway, and has a disadvantage in that the content is not so high. Korean Patent No. 10-2019448 describes a novel microalga with high lutein productivity, but has a disadvantage that the microalga is only a newly discovered strain.

Accordingly, the present inventors have made extensive efforts to solve the above-described problems and develop a strain capable of producing a large amount of lutein, and as a result, have found that, when a lutein biosynthetic pathway is introduced into a host cell having ability to produce farnesyl diphosphate, it is possible to produce a recombinant microorganism having ability to produce lutein, and when a substrate tunnel is constructed in the recombinant microorganism and an electron tunnel is produced in the recombinant microorganism and then the intracellular production of C5 heme is increased, the recombinant microorganism is capable of producing a large amount of lutein, thereby completing the present invention.

### SUMMARY OF THE INVENTION

An object of the present invention is to provide a recombinant strain capable of producing a large amount of lutein.

Another object of the present invention is to provide a method of producing lutein using the strain.

In order to achieve the above object, the present invention provides a recombinant microorganism having ability to produce lutein, which is obtained by introducing a lutein biosynthetic pathway into a host cell having ability to produce farnesyl diphosphate (FPP).

The present invention also provides a recombinant microorganism having enhanced ability to produce lutein, which is obtained by introducing a lutein biosynthetic pathway into a host cell having farnesyl diphosphate (FPP) and introducing any one or more selected from the group consisting of substrate tunnel formation, electron tunnel formation, and C5 heme production pathway modification.

The present invention also provides a method for producing lutein comprising steps of: (a) producing lutein by culturing the recombinant microorganism; and (b) recovering the produced lutein.

### BRIEF DESCRIPTION OF DRAWINGS

FIG. 1 shows the major metabolic pathways for lutein biosynthesis and the results of flask culture. FIG. 1(a) shows enzymes involved in an exogenous lutein biosynthetic pathway constructed in a cell according to the present invention. Bent arrows and the character T indicate promoters and transcription terminators, respectively. The abbreviations indicate the following: 5-ALA, 5-aminolevulinic acid; DXP, 1-deoxy-D-xylulose 5-phosphate; FPP, farnesyl diphosphate; L-Glu-tRNA, L-glutamyl-tRNA; G3P, D-glyceraldehyde 3-phosphate; GGPP, geranylgeranyl diphosphate; and TCA cycle, tricarboxylic acid cycle. FIG. 1(b) shows the results of flask culture of LUT1 to 4 and LUT4M strains. FIG. 1(c) schematically shows electron transfer from ATR2 to two P450s. It was expected that, when the CipB scaffold proteins linked to ATR2, trLUT5 and trLUT1 were brought together in the cell, electron transfer would occur more efficiently due to the closer distance therebetween. FIG. 1(d) shows the results of flask culture of LUT4M and LUT5M strains. Error bars mean standard deviation (n=3). **P<0.01; ***P<0.001. The abbreviations mean the following: ns, not significant.

FIG. 2 shows increased lutein production resulting from increased heme supply and culture condition optimization and the results of fed-batch culture of LUT5MH1. FIG. 2(a) shows the biosynthetic pathway of lutein and heme, and electron transfer from NADPH to oxygen via ATR2 and trLUT5/trLUT1. The abbreviations mean the following: 5-ALA, 5-aminolevulinic acid; DXP, 1-deoxy-D-xylulose 5-phosphate; L-Glu-tRNA, L-glutamyl-tRNA; G3P, D-glyceraldehyde 3-phosphate; and TCA cycle, tricarboxylic acid cycle. FIG. 2(b) shows the results of flask culture of LUT5M, LUT5MH1, and LUT5MH2 strains. FIG. 2(c) shows the result of comparison between R/2 medium and MR medium. The LUT5MH1 strain was cultured in R/2 or MR medium at 30° C., and then gene expression was induced with 1 mM IPTG. FIG. 2(d) shows the results of testing various culture temperatures for lutein production. The LUT5MH1 strain was cultured in R/2 medium at each of 37, 30, 28, 25 and 22° C., and gene expression was induced using 1 mM IPTG. FIG. 2(e) shows the results of comparing the effect of IPTG concentration, used for gene expression, on lutein production. LUT5MH1 was cultured in R/2 medium at 28° C., and when the OD₆₀₀ reached 0.4 to 0.6, IPTG was added to final concentrations of 0, 0.01, 0.02, 0.05, 0.1, 0.2, 0.5 and 1 mM. Error bars mean standard deviation (n=3). *P<0.05; **P <0.01; ***P<0.001. The abbreviations mean the following: ns, significant. FIG. 2(f) depicts photographs of the incubator during fed-batch culture of LUT5MH1. FIG. 2(g) shows the results of fed-batch culture of LUT5MH1. The culture temperature was maintained at 37° C. and then decreased to 28° C. after addition of 0.5 mM IPTG. In addition, carbon starvation was induced for 2 hours (at 12 to 14 hours) after depletion of the initially supplied carbon source.

FIG. 3 shows the results of analysis of the intracellular localization of enzymes linked to CipA or CipB, the intracellular formation of protein crystalline inclusions (PCIs), and verification of lutein production. FIG. 3(a) shows the HPLC-MS chromatograms and MS spectra of the lutein standard (molecular weight: 568.89 g mol⁻¹) and the LUT3 cell extract. The arrows indicate the lutein peaks corresponding to the MS spectra (m/z=551.4 [M+H-H20]⁻1. FIG. 3 (b) shows micrographs of WLGB-RPP strains having one or two of LUT1, pLUT2, pLUT3, pLUT4, pLUT5, pLUT6 and pLUT7, respectively. Black arrows indicate PCIs formed within cells.

FIG. 3(c) shows the results of SDS-PAGE performed to analyze the intracellular localization of ATR2, trLUT5 and trLUT1. Each lane corresponds to cytoplasmic protein (lane 1) and membrane protein (lane 2) of WLGB-RPP (pTrc99a), and cytoplasmic protein (lane 3) and (4) membrane protein (lane 4) of WLGB-RPP (pLUT3). The arrows in lane 3 indicate trLUT5 (63.82 kDa) and trLUT1 (56.69 kDa) in order from the top, and the arrow in lane 4 indicates ATR2 (78.91 kDa). FIG. 3(d) shows the results of SDS-PAGE analysis of PCIs formed by CipA. PCIs were isolated from strains having WLGB-RPP strains having pTac15k and pBBR1Tac (lane 1), pLUT1 and pLUT2 (lane 2), pLUT4 and pLUT5 (lane 3), pLUT4 and pLUT6 (lane 4), and pLUT1 and pLUT5 (lane 5), respectively, and loaded in the corresponding lanes. The arrow indicates CipA-CrtI (66.74 kDa). FIG. 3(e) shows the results of SDS-PAGE analysis of PCIs formed by CipB. PCIs were isolated from WLGB-RPP strains having pTrc99a (lane 1), pLUT3 (lane 2) and pLUT7 (lane 3), respectively, and loaded in the corresponding lanes. It appears that proteins in lane 2 include membrane proteins including ATR2 (78.91 kDa), as well as trLUT5 (63.82 kDa) and trLUT1 (56.69 kDa), which are originally cytoplasmic proteins, but are prematurely terminated, aggregated, or misfolded. The arrows in lane 3 indicate CipA-ATR2 (90.19 kDa), CipA-trLUT5 (75.09 kDa) and CipA-trLUT1 (67.96 kDa) in order from the top. Lane (L) in all SDS-PAGE results corresponds to protein size markers.

FIG. 4 shows the results of fed-batch culture of the LUT5MH1 strain under different temperature conditions and different IPTG concentration conditions. The culture temperature was maintained at 28° C. (FIG. 4a) or 37° C. (FIG. 4b), and then decreased to 28° C. after addition of 0.05 mM IPTG. For other graphs, the culture temperature was maintained at 37° C., and then decreased to 28° C. after addition of 0.2 mM (FIG. 4c), 0.5 mM (FIG. 4d and 4f), or 1 mM IPTG (FIG. 4e). In the fed-batch culture in FIG. 4(f), carbon starvation was induced for 2 hours after depletion of the initial carbon source.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

Definitions of key terms used in the detailed description of the present invention, etc. are as follows.

As used herein, the term “host cell” means any cell capable of expressing a functional gene and/or gene product derived from another cell or organ.

As used herein, the term “gene” is to be considered in its broadest sense, and the gene may encode a structural protein or a regulatory protein. In this case, examples of the regulatory protein include transcription factors, heat shock proteins, or proteins involved in DNA/RNA replication, transcription and/or translation.

As used herein, the term “lutein” refers to a compound having a molecular structure of Structural Formula 1 below.

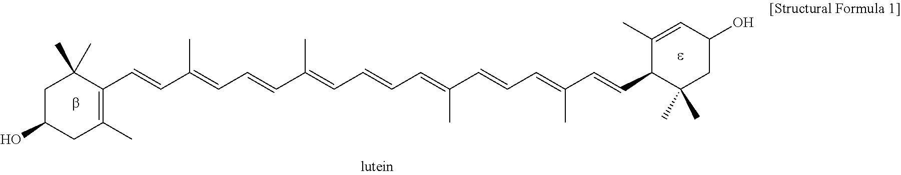

As used herein, the term “weakening” is meant to encompass reducing the activity of an enzyme, which is encoded by the gene of interest, by mutation, substitution or deletion of one or more nucleotides in the gene or by introduction of one or more nucleotides into the gene, and includes blocking a part or a significant part of a biosynthetic pathway in which the enzyme encoded by the gene is involved.

As used herein, the term “lacking” is meant to encompass preventing the gene of interest from being expressed or from showing enzymatic activity even if expressed, by mutation, substitution or deletion of some or all of the nucleotides in the gene or by introduction of some nucleotides into the gene, and includes blocking a biosynthetic pathway in which the enzyme encoded by the gene is involved.

As used herein, the term “amplification” is meant to encompass increasing the activity of an enzyme, which is encoded by the gene of interest, by mutation, substitution or deletion of one or more nucleotides in the gene, or introduction of one or more nucleotides into the gene, or introduction of a gene derived from another microorganism, which encodes the same enzyme.

In the present invention, examination was made to determine whether lutein was produced when a lutein biosynthetic pathway was introduced into an existing host cell having ability to produce farnesyl diphosphate. In addition, in order to develop a strain capable of producing lutein in large amounts by manipulating various metabolic pathways in the microorganism into which the lutein biosynthetic pathway has been introduced, examination was made, and as a result, it was confirmed that, when a substrate tunnel, electron tunnel, and C5 heme production pathway in the microorganism were manipulated, the microorganism could produce a large amount of lutein.

That is, in one example of the present invention, a lutein biosynthetic pathway was introduced into a WLGB-RPP strain having ability to produce lycopene (Choi, H S, et al., Appl Environ Microb 76, 3097-3105, 2010). Specifically, a recombinant E. coli platform strain having ability to produce lutein was constructed by inserting genes encoding enzymes derived from bacteria or eukaryotes.

In addition, various metabolic pathways in the E. coli platform strain were manipulated. There are two representative bottlenecks in the lutein biosynthetic pathway: promiscuous enzymes that are involved in metabolic flux; and two cytochrome P450 enzymes that are generally less active when expressed in microorganisms.

First, the problem caused by promiscuous enzymes was overcome by minimizing by-products through the creation of a substrate tunnel. The problem caused by P450 may occur due to the inefficiency of electron transfer between P450 and reductase, and taking this into consideration, an electron tunnel was created to facilitate electron transfer between the two enzymes.

In addition, when the intracellular production of heme, which is a cofactor of P450 acting as a mediator of electron transfer, was increased, P450 activity was further increased, thereby significantly increasing the production of lutein.

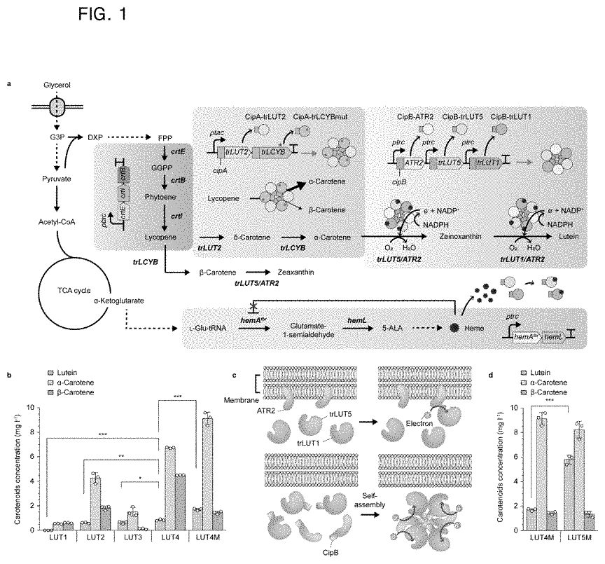

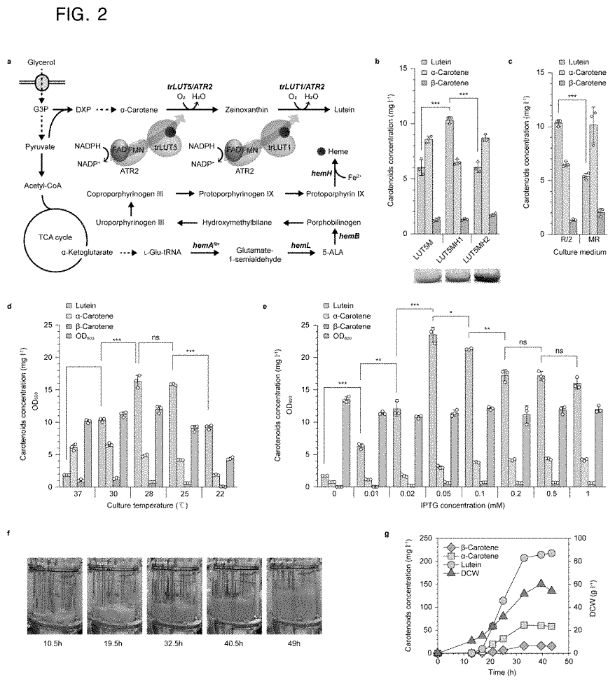

More specifically, it was confirmed that, when crtE, crtB, crtI, cipA-trLUT2, cipA-trLCYBmut, cipB-trLUT5, cipB-trLUT1, cipB-ATR2, hemA^{fbr} and hemL genes were introduced into a WLGB-RPP (ΔlacI ΔgdhA ΔgpmB ptrc dxs idi ispA pps) strain, the strain could produce lutein with the highest productivity (FIGS. 1 and 2).

Therefore, in one aspect, the present invention is directed to a recombinant microorganism having ability to produce lutein, which is obtained by introducing a lutein biosynthetic pathway into a host cell having ability to produce farnesyl diphosphate (FPP).

In the present invention, the host cell having ability to produce farnesyl diphosphate (FPP) may be a host cell which lacks at least one gene selected from the group consisting of lad (lactose operon repressor) gene, gdhA (NADP-specific glutamate dehydrogenase) gene and gpmB (phosphoglycerate mutase) gene and in which at least one gene selected from the group consisting of dxs (1-deoxyxylulose-5-phosphate synthase) gene, idi (isopentenyl diphosphate (IPDP) isomerase) gene, ispA (geranyltranstransferase/dimethylallyltranstransferase) gene and pps (PEP synthase) gene has been introduced or amplified.

More preferably, the host cell may be a WLGB-RPP strain, without being limited thereto.

In the present invention, the lutein biosynthetic pathway may be a lutein biosynthetic pathway into which at least one gene selected from the group consisting of crtE (geranylgeranyl pyrophosphate synthase) gene, crtB (phytoene synthase) gene, crtI (phytoene dehydrogenase) gene, LUT2 (lycopene ε-cyclase) gene, LCYB (lycopene β-cyclase) gene, ATR2 (cytochrome P450 reductase) gene, LUT5 (β-carotene 3-hydroxylase) gene and LUT1 (carotene ε-monooxygenase) gene has been introduced.

In the present invention, the LUT2 gene may be represented by the nucleotide sequence of SEQ ID NO: 19, without being limited thereto.

In the present invention, the LCYB gene may be represented by the nucleotide sequence of SEQ ID NO: 20, without being limited thereto.

In the present invention, the LUT5 gene may be represented by the nucleotide sequence of SEQ ID NO: 21, without being limited thereto.

In the present invention, the LUT1 gene may be represented by the nucleotide sequence of SEQ ID NO: 22, without being limited thereto.

In the present invention, the LCYB gene may encode G451E mutant protein.

In the present invention, when the LCYB gene encodes the G451E mutant protein, it may be represented by the nucleotide sequence of SEQ ID NO: 58, without being limited thereto.

In the present invention, the recombinant microorganism into which the lutein biosynthetic pathway has been introduced may be a recombinant microorganism into which any one or more selected from the group consisting of substrate tunnel formation, electron tunnel formation and C5 heme production pathway modification has been introduced so that the ability to produce lutein is further enhanced.

In the present invention, the substrate tunnel formation may be performed by introducing the cipA gene.

In the present invention, the introduction of the cipA gene may comprise modifying any one or more genes, selected from the group consisting of crtl, LUT2 and LCYB, into any one or more genes selected from the group consisting of cipA-crtl, cipA-LUT2 and cipA-LCYB.

In the present invention, the cipA-crtl gene may be represented by the nucleotide sequence of SEQ ID NO: 33, without being limited thereto.

In the present invention, the cipA-LUT2 gene may be represented by the nucleotide sequence of SEQ ID NO: 34, without being limited thereto.

In the present invention, the cipA-LCYB gene may be represented by the nucleotide sequence of SEQ ID NO: 35, without being limited thereto.

In the present invention, when the cipA-LCYB gene encodes a G451E mutant protein, it may be represented by the nucleotide sequence of SEQ ID NO: 36, without being limited thereto.

In the present invention, the electron tunnel formation may be performed by introducing the cipB gene.

In the present invention, the introduction of the cipB gene may comprise modifying any one or more genes, selected from the group consisting of ATR2, LUT5 and LUT1, into any one or more genes selected from the group consisting of cipB-ATR2, cipB-LUT5 and cipB-LUT1.

In the present invention, the cipB-ATR2 gene may be represented by the nucleotide sequence of SEQ ID NO: 41, without being limited thereto.

In the present invention, the cipB-LUT5 gene may be represented by the nucleotide sequence of SEQ ID NO: 42, without being limited thereto.

In the present invention, the cipB-LUT1 gene may be represented by the nucleotide sequence of SEQ ID NO: 43, without being limited thereto.

In the present invention, the C5 heme production pathway modification may be performed by introducing any one or more genes selected from the group consisting of hemA, hemL, hemB and hemH.

In the present invention, the hemA gene may encode a mutant protein that is resistant to feedback inhibition.

In the present invention, when the hemA gene encodes a mutant protein that is resistant to feedback inhibition, it may be represented by the nucleotide sequence of SEQ ID NO: 56, without being limited thereto.

In the present invention, the hemL gene may be represented by the nucleotide sequence of SEQ ID NO: 57, without being limited thereto.

As used herein, the term “vector” refers to a DNA construct containing a DNA sequence operably linked to a suitable regulatory sequence capable of expressing the DNA in a suitable host. The vector may be a plasmid, a phage particle, or simply a potential genomic insert. Once transformed into a suitable host, the vector may replicate and function independently of the host genome, or may in some instances, integrate into the genome itself. In the present specification, “plasmid” and “vector” are sometimes used interchangeably as the plasmid is the most commonly used form of vector at present. For the purposes of the present invention, a plasmid vector is preferably used. A typical plasmid vector that may be used for these purposes has a structure including: (a) a replication origin that allows effective replication so as to include several to hundreds of plasmid vectors per host cell; (b) an antibiotic resistance gene that enables selection of a host cell transformed with the plasmid vector; and (c) a restriction enzyme cleavage site into which a foreign DNA fragment may be inserted. Even if an appropriate restriction enzyme cleavage site is not present, the vector and foreign DNA can be easily ligated using a synthetic oligonucleotide adapter or a linker according to a conventional method. Even if an appropriate restriction enzyme cleavage site not present, the vector and the foreign DNA may be easily ligated using a synthetic oligonucleotide adapter or a linker according to a conventional method. After ligation, the vector is required to be transformed into the appropriate host cell. The transformation may be easily achieved by a calcium chloride method or electroporation (Neumann, et al., EMBO J., 1:841, 1982).

In the present invention, the nucleotide sequence is “operably linked” when it is placed in a functional relationship with another nucleic acid sequence. The term “operably linked” means that a gene and one or more transcriptional regulatory sequences are connected in such a way to permit gene expression when the appropriate molecules (e.g., transcriptional activator proteins) are bound to the regulatory sequences. For example, DNA for a pre-sequence or secretory leader is operably linked to DNA for a polypeptide if it is expressed as a pre-protein that participates in secretion of the polypeptide; a promoter or enhancer is operably linked to a coding sequence if it affects transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking between these sequences is accomplished by ligation at convenient restriction sites. If such sites do not exist, the synthetic oligonucleotide adaptors or linkers are used in accordance with conventional practice.

In the present invention, the host cell may be selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhodobacter, Zymomonas, Bacillus, Staphylococcus, Lactococcus, Streptococcus, Lactobacillus, Clostridium, Corynebacterium, Streptomyces, Bifidobacterium, Cyanobacterium, and Cyclobacterium.

In another aspect, the present invention is directed to a recombinant microorganism having enhanced ability to produce lutein, which is obtained by introducing a lutein biosynthetic pathway into a host cell having ability to produce farnesyl diphosphate (FPP) and introducing at least one selected from the group consisting of substrate tunnel formation, electron tunnel formation, and C5 heme production pathway modification.

In the present invention, the host cell having ability to produce farnesyl diphosphate (FPP) may be a host cell which lacks at least one gene selected from the group consisting of lad (lactose operon repressor) gene, gdhA (NADP-specific glutamate dehydrogenase) gene and gpmB (phosphoglycerate mutase) gene and in which at least one gene selected from the group consisting of dxs (1-deoxyxylulose-5-phosphate synthase) gene, idi (isopentenyl diphosphate (IPDP) isomerase) gene, ispA (geranyltranstransferase/dimethylallyltranstransferase) gene and pps (PEP synthase) gene has been introduced or amplified.

More preferably, the host cell may be a WLGB-RPP strain, without being limited thereto.

In the present invention, the lutein biosynthetic pathway may be a lutein biosynthetic pathway into which at least one gene selected from the group consisting of crtE (geranylgeranyl pyrophosphate synthase) gene, crtB (phytoene synthase) gene, crtI (phytoene dehydrogenase) gene, LUT2 (lycopene ε-cyclase) gene, LCYB (lycopene β-cyclase) gene, ATR2 (cytochrome P450 reductase) gene, LUT5 (β-carotene 3-hydroxylase) gene and LUT1 (carotene ε-monooxygenase) gene has been introduced.

In the present invention, the LUT2 gene may be represented by the nucleotide sequence of SEQ ID NO: 19, without being limited thereto.

In the present invention, the LCYB gene may be represented by the nucleotide sequence of SEQ ID NO: 20, without being limited thereto.

In the present invention, the LUT5 gene may be represented by the nucleotide sequence of SEQ ID NO: 21, without being limited thereto.

In the present invention, the LUT1 gene may be represented by the nucleotide sequence of SEQ ID NO: 22, without being limited thereto.

In the present invention, the LCYB gene may encode G451E mutant protein.

In the present invention, when the LCYB gene encodes the G451E mutant protein, it may be represented by the nucleotide sequence of SEQ ID NO: 58, without being limited thereto.

In the present invention, the substrate tunnel formation may be performed by introducing the cipA gene.

In the present invention, introduction of the cipA gene may comprise modifying any one or more genes, selected from the group consisting of crtl, LUT2 and LCYB, into any one or more genes selected from the group consisting of cipA-crtl, cipA-LUT2 and cipA-LCYB.

In the present invention, the cipA-crtl gene may be represented by the nucleotide sequence of SEQ ID NO: 33, without being limited thereto.

In the present invention, the cipA-LUT2 gene may be represented by the nucleotide sequence of SEQ ID NO: 34, without being limited thereto.

In the present invention, the cipA-LCYB gene may be represented by the nucleotide sequence of SEQ ID NO: 35, without being limited thereto.

In the present invention, when the cipA-LCYB gene encodes a G451E mutant protein, it may be represented by the nucleotide sequence of SEQ ID NO: 36, without being limited thereto.

In the present invention, the electron tunnel formation may be performed by introducing the cipB gene.

In the present invention, the introduction of the cipB gene may comprise modifying any one or more genes, selected from the group consisting of ATR2, LUT5 and LUT1, into any one or more genes selected from the group consisting of cipB-ATR2, cipB-LUT5 and cipB-LUT1.

In the present invention, the cipB-ATR2 gene may be represented by the nucleotide sequence of SEQ ID NO: 41, without being limited thereto.

In the present invention, the cipB-LUT5 gene may be represented by the nucleotide sequence of SEQ ID NO: 42, without being limited thereto.

In the present invention, the cipB-LUT1 gene may be represented by the nucleotide sequence of SEQ ID NO: 43, without being limited thereto.

In the present invention, the C5 heme production pathway modification may be performed by introducing any one or more genes selected from the group consisting of hemA, hemL, hemB and hemH.

In the present invention, the hemA gene may encode a mutant protein that is resistant to feedback inhibition.

In the present invention, when the hemA gene encodes a mutant protein that is resistant to feedback inhibition, it may be represented by the nucleotide sequence of SEQ ID NO: 56, without being limited thereto.

In the present invention, the hemL gene may be represented by the nucleotide sequence of SEQ ID NO: 57, without being limited thereto.

In still another aspect, the present invention is directed to a method for producing lutein comprising steps of: (a) producing lutein by culturing the recombinant microorganism; and (b) recovering the produced lutein.

In the present invention, the process of culturing the recombinant microorganism and recovering lutein may be performed using a culture method (batch culture, or fed-batch culture) and a lutein separation and purification method commonly known in a conventional fermentation process.

In the present invention, biotechnological production of lutein may be performed intracellularly or extracellularly (in vivo or in vitro).

In the present invention, step (a) may comprise a step of making the temperature of the culturing after expression of the lutein biosynthetic pathway genes different from the temperature of the culturing before the expression, and inducing carbon starvation after the expression.

Preferably, the step of producing lutein by culturing the recombinant microorganism may comprise the following steps, without being limited thereto.

(a) culturing the recombinant microorganism in R/2 medium at 37° C.;

(b) when the OD₆₀₀ value reaches 20 to 30, injecting IPTG to a final concentration of 0.5 mM, changing the temperature of the culturing to 28° C., and culturing the recombinant microorganism; and

(c) culturing the recombinant microorganism without supplying a carbon source for 2 hours after depletion of the initial carbon source.

In the present invention, the method may further comprise, after step (c), a step of additionally injecting a carbon source and culturing the recombinant microorganism.

### EXAMPLES

Hereinafter, the present invention will be described in more detail with reference to specific examples. However, the present invention is not limited by the following examples, and it will be apparent to those skilled in the art that various changes or modifications may be made within the idea and scope of the present invention.

### Example 1. Construction of Lutein Production Pathway

1-1. Selection of Lutein Biosynthetic Pathway Genes

First, crtE, crtB and crtI from Pantoea ananatis, which are genes producing lycopene, a precursor of lutein, were introduced into a pTac15k plasmid, thereby constructing pLUT1. Next, in order to convert lycopene to α-carotene, LUT2 and LCYB from Arabidopsis thaliana, which were codon-optimized and from which the N-terminal signal peptide coding sequence was cut out, were introduced into a pBBR1Tac plasmid, thereby constructing pLUT2. The N-terminal signal peptide sequence was predicted using ChloroP software (Emanuelsson, O., Nielsen, et al., Protein Sci 8, 978-984, 1999).

Finally, in order to convert alpha-carotene into lutein, LUT5 and LUT1 from A. thaliana, which were codon-optimized and from which the N-terminal signal peptide coding sequence was cut out, were introduced into a pTrc99a plasmid. LUT5 and LUT1 are cytochrome P450 enzymes (P450) and require a partner reductase to have activity in E. coli. Thus, ATR2 from A. thaliana was codon-optimized and also introduced, thereby constructing pLUT3. ATR2 also has a putative signal peptide sequence at the N-terminus, but the signal peptide sequence was not removed, in consideration of reports that the activity was higher when the entire sequence was expressed in E. coli. FIG. 1a shows the major lutein biosynthetic pathway.

1-2. Plasmid Construction

A more detailed plasmid construction procedure is as follows. First, either DNA digestion using restriction enzymes or Gibson's method (Gibson, D. G. et al. Nat Methods 6, 343-U341, 2009) was used to construct the plasmids in this study. crtE, crtI and crtB from P. ananatis were used after amplification from pCar184 (Choi, H. S., et al., Appl Environ Microb 76, 3097-3105, 2010), and trLUT2, trLCYB, ATR2, trLUT5 and trLUT1 genes from A. thaliana were synthesized and used after codon optimization.

To construct pLUT1, crtE and crtl-crtB from pCar184 were amplified using a combination of crtE_F/crtE_R1 primers and a combination of crtI_F1/crtB_R primers, respectively, and then ligated together by PCR and inserted into the SacI/XbaI cleavage sites of pTac15k. To construct pLUT2, trLUT2 and trLCYB genes were amplified using a combination of trLUT2_F/R1 primers and a combination of trLCYB_F/R primers, respectively, and then inserted into the SacI/XbaI and XbaI/PstI cleavage sites of the pBBR1Tac plasmid, respectively.

Before construction of pLUT3, pTrc99a plasmids expressing each of ATR2, trLUT5 and trLUT1 were constructed. The genes were amplified using ATR2_F1/R1, trLUT5_F/R1, and trLUT1_F/R1 primer sets, respectively, and then inserted into EcoRI/BamHI cleavage sites of pTrc99a plasmids, thereby constructing pTrc-ATR2, pTrc-trLUT5, and pTrc-trLUT1, respectively. The trLUT5 gene ligated with the trc promoter was amplified from pTrc-trLUT5 using pTrc_F1/trLUT5_R2 primers, and the trLUT1 gene ligated with the trc promoter was amplified using pTrc_F2/trLUT1_R2 primers. Then, the amplified genes were inserted into the BamHI/XbaI and XbaI/SalI cleavage sites of the pTrc-ATR2 plasmid, respectively, thereby constructing pLUT3.

Table 1 below shows the sequences of the primers used in plasmid construction.

TABLE 1

| SEQ | | |
|---|---|---|
| ID | | |
| NO | Name | Sequence |
| 1 | crtE_F | AGACAGGAGCTCTTCACACAGGAAACAATGTATC |
| | | CGTTTATAAGGAC |
| 2 | crtE_R1 | TTAACTGACGGCAGCGAGTT |
| 3 | crtI_F1 | AACTCGCTGCCGTCAGTTAATTCACACAGGAAAC |
| | | AATGAAACCAACTACGGTAAT |
| 4 | crtB_R | AGACAGTCTAGATTAGAGCGGGCGCTGCCAGA |
| 5 | trLUT2_F | AGACAGGAGCTCTTCACACAGGAAACAATGGCAT |
| | | CCGGCGGT |
| 6 | trLUT2_R | AGACAGTCTAGATTACACTTTCAGGTAGGTTTTG |
| 7 | trLCYB_F | AGACAGTCTAGATTCACACAGGAAACAATGGTCG |
| | | TGTCGGGG |
| 8 | trLCYB_R | AGACAGCTGCAGTTAGTCGCGATCCTGCACTA |
| 9 | ATR2_F1 | AGACAGGAATTCTTCACACAGGAAACAATGTCTT |
| | | CTTCTTCTTCTTC |
| 10 | ATR2_R1 | AGACAGGGATCCTTACCACACATCACGAAGAT |
| 11 | trLUT5_F | AGACAGGAATTCTTCACACAGGAAACAATGTTCA |
| | | GCAGCTCAAG |
| 12 | trLUT5_R1 | AGACAGGGATCCTTAAGAAAGGGCCGAGCTAA |
| 13 | trLUT1_F | AGACAGGAATTCTTCACACAGGAAACAATGAGTT |
| | | CCATCGAAAAACC |
| 14 | trLUT1_R1 | AGACAGGGATCCTTAACGTTGAGAAACTTTCA |
| 15 | pTrc_F1 | AGACAGGGATCCTTGACAATTAATCATCCGG |
| 16 | trLUT5_R2 | AGACAGTCTAGATTAAGAAAGGGCCGAG |
| 17 | pTrc_F2 | AGACAGTCTAGATTGACAATTAATCATCCGG |
| 18 | trLUT1_R2 | AGACAGGTCGACTTAACGTTGAGAAACTTTCA |

TABLE 2
Codon-optimized gene sequences

| SEQ ID | | |
|---|---|---|
| NO | Name | Sequence |
| 19 | trLUT2 | ATGGCATCCGGCGGTGGCAGCTCTGGCAGCGAATCTTGTGTGG |
| | | CGGTTCGCGAAGACTTTGCCGATGAAGAAGACTTCGTCAAAGC |
| | | CGGTGGCTCTGAAATCCTGTTTGTGCAGATGCAGCAAAACAAA |
| | | GACATGGATGAACAAAGTAAACTGGTTGATAAACTGCCGCCGA |
| | | TTTCCATCGGTGATGGCGCGCTGGACCTGGTTGTGATTGGTTGT |
| | | GGTCCGGCCGGTCTGGCACTGGCAGCTGAAAGCGCCAAACTGG |
| | | GCCTGAAAGTCGGTCTGATCGGCCCGGATCTGCCGTTTACCAAC |
| | | AATTATGGTGTGTGGGAAGATGAATTTAATGACCTGGGCCTGC |
| | | AAAAATGCATTGAACATGTGTGGCGTGAAACGATCGTTTATCTG |
| | | GATGACGATAAACCGATTACCATTGGTCGTGCGTACGGCCGTGT |
| | | GAGCCGTCGCCTGCTGCACGAAGAACTGCTGCGTCGCTGTGTTG |
| | | AATCAGGTGTCTCGTATCTGAGTTCCAAAGTTGATAGTATTACG |
| | | GAAGCATCCGATGGTCTGCGTCTGGTCGCTTGTGACGATAACAA |
| | | TGTGATTCCGTGTCGCCTGGCTACCGTGGCGTCAGGTGCCGCCT |
| | | CGGGTAAACTGCTGCAATATGAAGTTGGTGGCCCGCGTGTCTGT |
| | | GTGCAAACCGCGTATGGTGTTGAAGTCGAAGTGGAAAACTCAC |
| | | CGTACGACCCGGATCAGATGGTTTTTATGGATTATCGTGACTAC |
| | | ACCAATGAAAAAGTCCGCAGCCTGGAAGCGGAATATCCGACCT |
| | | TCCTGTACGCCATGCCGATGACGAAATCACGCCTGTTTTTCGAA |
| | | GAAACCTGTCTGGCCTCGAAAGATGTGATGCCGTTTGACCTGCT |
| | | GAAAACCAAACTGATGCTGCGTCTGGATACGCTGGGCATTCGC |
| | | ATCCTGAAAACCTATGAAGAAGAATGGTCATACATTCCGGTTG |
| | | GTGGCTCGCTGCCGAACACGGAACAGAAAAATCTGGCATTTGG |
| | | TGCAGCTGCGAGCATGGTCCATCCGGCTACCGGCTATTCTGTTG |
| | | TCCGTAGTCTGTCCGAAGCCCCGAAATACGCAAGTGTTATTGCT |
| | | GAAATCCTGCGTGAAGAAACCACGAAACAGATTAACAGCAATA |
| | | TCTCTCGCCAAGCGTGGGATACCCTGTGGCCGCCAGAACGTAA |
| | | ACGCCAGCGTGCGTTTTTCCTGTTTGGTCTGGCCCTGATTGTGC |
| | | AATTCGATACGGAAGGCATCCGCAGCTTTTTCCGTACCTTTTTC |
| | | CGCCTGCCGAAATGGATGTGGCAGGGTTTTCTGGGCAGCACCCT |
| | | GACGTCTGGCGATCTGGTTCTGTTTGCACTGTATATGTTCGTCAT |
| | | TAGCCCGAACAATCTGCGCAAAGGTCTGATTAACCACCTGATCT |
| | | CTGATCCGACCGGCGCTACGATGATCAAAACCTACCTGAAAGT |
| | | GTAA |
| 20 | trLCYB | ATGGTCGTGTCGGGGAGCGCGGCCTTGCTGGATTTAGTCCCCGA |
| | | AACCAAGAAAGAGAACCTGGACTTTGAGTTACCCCTTTATGAT |
| | | ACGAGTAAATCCCAAGTTGTCGATCTGGCAATCGTCGGAGGGG |
| | | GCCCGGCAGGGTTAGCAGTGGCCCAGCAAGTAAGTGAGGCCGG |
| | | TCTTAGCGTGTGCTCGATTGACCCCAGTCCCAAGTTGATCTGGC |
| | | CCAACAATTATGGAGTATGGGTCGATGAATTTGAAGCTATGGA |
| | | CCTGTTAGACTGCCTGGATACTACTTGGAGTGGAGCAGTTGTGT |
| | | ATGTAGATGAAGGAGTTAAAAAAGATCTGTCACGTCCTTATGG |
| | | ACGTGTAAACCGCAAACAGTTAAAGTCAAAGATGCTGCAAAAA |
| | | TGTATCACGAACGGTGTGAAGTTTCATCAGTCCAAAGTGACCA |
| | | ATGTAGTTCACGAGGAGGCAAATTCAACGGTTGTCTGTAGCGA |
| | | CGGTGTGAAAATCCAAGCCAGCGTAGTCCTGGATGCGACGGGG |
| | | TTTTCGCGTTGCCTGGTTCAATACGACAAGCCCTACAACCCAGG |
| | | CTACCAAGTTGCCTACGGAATTGTAGCAGAAGTAGATGGGCAC |
| | | CCCTTCGACGTTGACAAAATGGTTTTTATGGACTGGCGTGATAA |
| | | GCACCTGGATAGTTACCCAGAATTGAAGGAGCGCAATTCAAAA |
| | | ATTCCTACATTCCTTTATGCGATGCCTTTCTCGTCCAATCGCATC |
| | | TTCTTGGAGGAGACCTCCCTGGTAGCTCGCCCGGGTCTTCGCAT |
| | | GGAAGATATCCAGGAACGCATGGCCGCTCGCTTAAAGCACTTA |
| | | GGTATCAACGTGAAGCGTATTGAAGAAGATGAACGCTGTGTAA |
| | | TCCCCATGGGTGGGCCACTGCCCGTGTTACCTCAACGTGTGGTA |
| | | GGTATCGGTGGCACGGCCGGCATGGTTCATCCTAGTACCGGTTA |
| | | CATGGTAGCCCGCACACTGGCAGCGGCACCTATTGTCGCAAAT |
| | | GCAATCGTTCGTTATTTGGGTTCCCCCTCGTCAAACTCCTTACGC |
| | | GGAGACCAGTTAAGTGCCGAAGTTTGGCGCGACCTTTGGCCCA |
| | | TCGAACGTCGTCGCCAACGCGAATTCTTCTGTTTTGGGATGGAT |
| | | ATCTTATTAAAACTGGACTTAGATGCGACCCGCCGCTTCTTCGA |
| | | CGCATTCTTCGATCTTCAGCCACACTACTGGCACGGATTTTTAT |
| | | CATCGCGTTTATTTCTGCCCGAGTTATTGGTATTCGGCTTGAGTC |
| | | TTTTTTCTCATGCGTCGAACACTTCCCGCCTGGAGATCATGACT |
| | | AAAGGCACCGTACCATTGGCTAAGATGATCAATAACTTAGTGC |
| | | AGGATCGCGACTAA |
| 21 | trLUT5 | ATGTTCAGCAGCTCAAGTAACGGCCGCGATCCACTTGAGGAAA |
| | | ACTCAGTACCGAATGGCGTGAAAAGCCTGGAGAAATTGCAGGA |
| | | GGAAAAACGCCGTGCTGAATTGTCGGCCCGTATCGCATCCGGT |
| | | GCATTCACCGTTCGTAAGAGCAGCTTCCCCAGCACTGTCAAGAA |
| | | TGGTCTTAGCAAAATCGGGATTCCTAGCAACGTATTGGATTTTA |
| | | TGTTCGACTGGACAGGAAGTGACCAGGACTATCCAAAAGTACC |
| | | TGAGGCGAAGGGGTCAATTCAAGCTGTTCGCAACGAGGCGTTT |
| | | TTTATTCCGCTTTACGAACTTTTTTTAACTTATGGTGGGATCTTT |
| | | CGCCTGACATTTGGCCCTAAAAGCTTCCTTATTGTTAGCGATCC |
| | | CTCAATCGCCAAACATATCCTGAAGGATAACGCCAAAGCTTAC |
| | | TCCAAGGGCATTTTAGCGGAAATCTTAGACTTCGTGATGGGTAA |
| | | AGGTCTGATCCCGGCCGATGGCGAGATTTGGCGTCGCCGTCGCC |
| | | GTGCTATCGTGCCGGCTCTTCATCAGAAGTATGTCGCTGCAATG |
| | | ATCAGCCTTTTCGGTGAAGCCTCAGATCGTTTGTGTCAGAAGTT |
| | | AGACGCGGCAGCCTTGAAAGGGGAGGAGGTCGAAATGGAGAG |
| | | CCTGTTCTCACGTTTAACTTTGGACATTATTGGTAAAGCCGTATT |
| | | TAATTATGACTTTGACTCCCTTACCAACGACACGGGGGTGATTG |
| | | AGGCTGTCTACACGGTGCTGCGCGAAGCAGAAGACCGTTCTGT |
| | | TAGCCCTATCCCGGTTTGGGATATCCCTATTTGGAAAGACATCT |
| | | CTCCGCGTCAGCGTAAAGTTGCAACAAGCTTGAAATTAATTAAC |
| | | GACACACTTGATGACTTAATCGCAACATGTAAGCGTATGGTTGA |
| | | GGAAGAAGAGTTACAGTTTCATGAGGAATATATGAACGAACGT |
| | | GATCCGTCCATTTTGCATTTTTTACTTGCTAGTGGTGACGACGTC |
| | | AGCTCCAAACAGTTACGTGACGACCTGATGACTATGCTGATCGC |
| | | TGGTCATGAAACATCAGCAGCTGTGTTGACCTGGACATTTTACT |
| | | TGCTTACAACGGAGCCGTCAGTGGTGGCGAAGCTTCAGGAGGA |
| | | GGTGGACTCCGTTATTGGAGACCGCTTCCCTACAATTCAAGACA |
| | | TGAAGAAATTAAAATATACAACTCGCGTTATGAACGAATCTCT |
| | | GCGCTTGTATCCCCAACCACCTGTTTTAATTCGTCGCTCTATTGA |
| | | CAATGACATCTTAGGCGAATACCCTATCAAGCGTGGTGAGGAC |
| | | ATCTTCATTTCTGTGTGGAATTTGCATCGCAGCCCCTTGCACTG |
| | | GGATGATGCGGAAAAATTTAATCCCGAGCGTTGGCCACTGGAT |
| | | GGTCCCAACCCAAATGAGACGAATCAGAATTTCAGCTATTTGCC |
| | | TTTTGGCGGAGGACCGCGTAAGTGTATTGGAGATATGTTCGCCA |
| | | GCTTTGAGAATGTGGTAGCCATCGCCATGCTGATCCGTCGTTTT |
| | | AACTTTCAAATTGCACCAGGAGCGCCCCCCGTAAAGATGACCA |
| | | CCGGTGCTACTATTCATACGACCGAAGGCTTGAAATTAACAGTA |
| | | ACGAAACGTACAAAACCATTAGATATTCCGTCCGTTCCGATCCT |
| | | TCCTATGGACACTTCGCGCGATGAAGTTAGCTCGGCCCTTTCTT |
| | | AA |
| 22 | trLUT1 | ATGAGTTCCATCGAAAAACCGAAACCGAAACTGGAAACCAACT |
| | | CATCGAAAAGTCAGTCCTGGGTGTCTCCGGATTGGCTGACCACC |
| | | CTGACCCGTACCCTGAGCAGCGGCAAAAACGACGAATCCGGTA |
| | | TTCCGATCGCAAATGCTAAACTGGATGATGTTGCGGATCTGCTG |
| | | GGCGGCGCGCTGTTTCTGCCGCTGTATAAATGGATGAACGAAT |
| | | ATGGTCCGATTTACCGCCTGGCGGCCGGTCCGCGTAATTTTGTC |
| | | ATTGTGAGCGATCCGGCGATCGCCAAACATGTGCTGCGTAACT |
| | | ATCCGAAATACGCCAAAGGCCTGGTGGCAGAAGTTAGCGAATT |
| | | TCTGTTCGGCTCTGGTTTTGCAATTGCTGAAGGTCCGCTGTGGA |
| | | CCGCCCGTCGCCGTGCAGTGGTTCCGAGTCTGCACCGCCGTTAC |
| | | CTGTCCGTTATCGTCGAACGCGTGTTCTGCAAATGTGCGGAACG |
| | | TCTGGTTGAAAAACTGCAACCGTATGCTGAAGATGGCAGTGCG |
| | | GTGAATATGGAAGCCAAATTTTCCCAAATGACCCTGGACGTTAT |
| | | CGGTCTGTCACTGTTCAACTACAACTTCGATTCACTGACCACGG |
| | | ACTCGCCGGTCATCGAAGCAGTGTACACCGCTCTGAAAGAAGC |
| | | GGAACTGCGCTCTACGGATCTGCTGCCGTATTGGAAAATTGACG |
| | | CACTGTGCAAAATCGTTCCGCGCCAGGTCAAAGCAGAAAAAGC |
| | | TGTCACCCTGATTCGTGAAACGGTGGAAGACCTGATTGCGAAA |
| | | TGTAAAGAAATCGTGGAACGCGAAGGCGAACGTATTAACGATG |
| | | AAGAATACGTTAATGATGCGGACCCGAGCATCCTGCGCTTCCTG |
| | | CTGGCCTCTCGTGAAGAAGTCAGTTCCGTGCAGCTGCGCGATGA |
| | | CCTGCTGTCTATGCTGGTCGCCGGCCATGAAACCACGGGTTCAG |
| | | TGCTGACCTGGACGCTGTATCTGCTGTCGAAAAACTCATCGGCC |
| | | CTGCGTAAAGCACAAGAAGAAGTTGATCGCGTCCTGGAAGGTC |
| | | GTAACCCGGCCTTCGAAGACATTAAAGAACTGAAATACATCAC |
| | | CCGCTGCATCAATGAAAGTATGCGTCTGTACCCGCATCCGCCGG |
| | | TTCTGATTCGCCGTGCACAGGTCCCGGATATTCTGCCGGGCAAC |
| | | TATAAAGTGAATACCGGTCAAGACATTATGATCTCCGTTTACAA |
| | | TATCCACCGCAGCTCTGAAGTCTGGGAAAAAGCAGAAGAATTT |
| | | CTGCCGGAACGTTTCGATATTGACGGCGCTATCCCGAACGAAA |
| | | CCAACACGGATTTCAAATTCATCCCGTTTAGCGGCGGTCCGCGC |
| | | AAATGTGTGGGTGATCAGTTCGCTCTGATGGAAGCGATCGTGG |
| | | CGCTGGCCGTGTTTCTGCAACGTCTGAACGTGGAACTGGTTCCG |
| | | GATCAAACCATTAGCATGACCACGGGCGCCACGATCCACACCA |
| | | CGAATGGTCTGTATATGAAAGTTTCTCAACGTTAA |

1-3. Analysis of Lutein Production

An LUT1 strain was constructed by introducing the pLUT1, pLUT2 and pLUT3 plasmids into the WLGB-RPP strain (Choi, H. S., et al., Appl Environ Microb 76, 3097-3105, 2010) constructed by the present inventors in the study of lycopene production. Glycerol known to be superior to glucose in previous carotenoid production studies was used as a carbon source, and expression of foreign genes was induced using 1 mM of isopropyl β-D-1-thiogalactopyranoside (IPTG).

As a result of flask culture, it was confirmed that only alpha-carotene (0.54 mg l⁻¹) and beta-carotene (0.61 mg l⁻¹) were produced, and lutein was not produced (FIG. 1b).

Strain name: LUT1

Genetic status: W3110 (ΔlacI ΔgdhA ΔgpmB ptrc dxs idi ispA pps), crtE, crtB, crtI, trLUT2, trLCYB, trLUT5, trLUT1, and ATR

### Example 2. Construction of Substrate Tunnel

2-1. Selection of Genes for Constructing Substrate Tunnel

The reason why lutein was not produced in Example 1 was believed to be because the promiscuous enzyme LCYB reduced the metabolic flux to the lutein pathway by converting lycopene into beta-carotene. Thus, to reduce the metabolic flux to the beta-carotene pathway and to increase the metabolic flux to the alpha-carotene, three substrate tunnels were designed using the CipA scaffold protein. The first substrate tunnel is a substrate tunnel that is based on CipA and links phytoene to alpha-carotene by bringing CrtI, trLUT2 and trLCYB together, the second is a substrate tunnel that links phytoene to delta-carotene (δ-carotene) by bringing CrtI and trLUT2 together, and the third is a substrate tunnel that links lycopene to alpha-carotene by bringing trLUT2 and trLCYB together.

It is known that CipA from Photorhabdus luminescens produces protein crystalline inclusions (PCIs) with enzymatic activity in E. coli, and that PCIs are formed in the same way even when CipA is fused with other enzymes (Wang, Y., et al., Acs Synth Biol 6, 826-836, 2017). Thus, pLUT4 was constructed by replacing crtI of pLUT1 with cipA-crtI, and pLUT5 was constructed by replacing trLUT2 and trLCYB of pLUT2 with trLUT2 and trLCYB, respectively. In addition, pLUT6 was also constructed by replacing only trLUT2 of pLUT2 with cipA-trLUT2.

2-2. Plasmid Construction

A detailed plasmid construction procedure is as follows. cipA from P. luminescens was synthesized and used after codon optimization. To construct pLUT4, cipA and crtI were amplified using a combination of cipA_F1/cipA_R1 primers and a combination of crtI_F2/crtI_R primers, respectively, and then ligated together by PCR and inserted into the crtI-inserted position of pLUT1 by Gibson's method. The plasmid was linearized by PCR amplification using crtB_F/crtE_R2 primers.

To construct pLUT5, cipA was amplified with cipA_F2/R2 and cipA_F3/R3 primers, and then ligated by PCR to the trLUT2 and trLCYB gene fragments used in the construction of pLUT2. The cipA-trLUT2 and cipA-trLCYB gene fragments were inserted into the SacI/XbaI and XbaI/PstI cleavage sites of the pBBR1Tac plasmid, respectively, thereby constructing pLUT5. pLUT6 was constructed by inserting and amplifying the cipA-trLUT2 and trLCYB gene fragments in the same manner.

Table 3 below the sequences of the primers used in construction of the plasmids.

TABLE 4

| SEQ ID | | |
|---|---|---|
| NO | Name | Sequence |
| 23 | cipA_F1 | AACTCGCTGCCGTCAGTTAATTCACACAG |
| | | GAAACAATGATTAATGACATGCATCCTT |
| 24 | cipA_R1 | CAATTACCGTAGTTGGTTTCATCATAGAG |
| | | ATTTCTACGCAATTA |
| 25 | crtI_F2 | ATGAAACCAACTACGGTAAT |
| 26 | crtI_R | TAAATCAGATCCTCCAGCATCA |
| 27 | crtB_F | TGATGCTGGAGGATCTGATTTAATTCACAC |
| | | AGGAAACAATGAATAATCCGTCGTTACTCA |
| 28 | crtE_R2 | TTAACTGACGGCAGCGAGTT |
| 29 | cipA_F2 | AGACAGGAGCTCTTCACACAGGAAACAATG |
| | | ATTAATGACATGCATCC |
| 30 | cipA_R2 | GCTGCCACCGCCGGATGCCATCATAGAGAT |
| | | TTCTACGCAATTA |
| 31 | cipA_F3 | AGACAGTCTAGATTCACACAGGAAACAATG |
| | | ATTAATGACATGCATCC |
| 32 | cipA_R3 | GCTCCCCGACACGACCATCATAGAGATTTC |
| | | TACGCAATTA |

TABLE 4
Gene sequences for constructing substrate
tunnel

| SEQ ID NO | Name | Sequence |
|---|---|---|
| 33 | cipA-crt1 | ATGATTAATGACATGCATCCTTCTTTAATTAAAGATAAAGATATAG |
| | | TAGATGACGTAATGCTACGTAGCTGTAAAATCATTGCAATGAAAGT |
| | | TATGCCAGACAAAGTTATGCAAGTTATGGTGACTGTATTAATGCAT |
| | | GATGGCGTATGTGAAGAAATGCTTTTGAAATGGAATCTGCTAGACA |
| | | ATAGAGGCATGGCGATTTATAAAGTTCTGATGGAAGCGCTTTGCGC |
| | | TAAGAAAGATGTGAAAATTAGCACCGTAGGAAAAGTAGGTCCTCTC |
| | | GGCTGCGATTACATTAATTGCGTAGAAATCTCTATGATGAAACCAA |
| | | CTACGGTAATTGGTGCAGGCTTCGGTGGCCTGGCACTGGCAATTCG |
| | | TCTACAGGCTGCGGGGATCCCCGTCTTACTGCTTGAACAACGTGAT |
| | | AAACCCGGCGGTCGGGCTTATGTCTACGAGGATCAGGGGTTTACCT |
| | | TTGATGCAGGCCCGACGGTTATCACCGATCCCAGTGCCATTGAAGA |
| | | ACTGTTTGCACTGGCAGGAAAACAGTTAAAAGAGTATGTCGAACTG |
| | | CTGCCGGTTACGCCGTTTTACCGCCTGTGTTGGGAGTCAGGGAAGG |
| | | TCTTTAATTACGATAACGATCAAACCCGGCTCGAAGCGCAGATTCA |
| | | GCAGTTTAATCCCCGCGATGTCGAAGGTTATCGTCAGTTTCTGGACT |
| | | ATTCACGCGCGGTGTTTAAAGAAGGCTATCTGAAGCTCGGTACTGT |
| | | CCCTTTTTTATCGTTCAGAGACATGCTTCGCGCCGCACCTCAACTGG |
| | | CGAAACTGCAGGCATGGAGAAGCGTTTACAGTAAGGTTGCCAGTTA |
| | | CATCGAAGATGAACATCTGCGCCAGGCGTTTTCTTTCCACTCGCTGT |
| | | TGGTGGGCGGCAATCCCTTCGCCACCTCATCCATTTATACGTTGATA |
| | | CACGCGCTGGAACGTGAGTGGGGCGTCTGGTTTCCGCGTGGCGGCA |
| | | CCGGCGCATTAGTTCAGGGGATGATAAAGCTGTTTCAGGATCTGGG |
| | | TGGCGAAGTCGTGTTAAACGCCAGAGTCAGCCATATGGAAACGAC |
| | | AGGAAACAAGATTGAAGCCGTGCATTTAGAGGACGGTCGCAGGTT |
| | | CCTGACGCAAGCCGTCGCGTCAAATGCAGATGTGGTTCATACCTAT |
| | | CGCGACCTGTTAAGCCAGCACCCTGCCGCGGTTAAGCAGTCCAACA |
| | | AACTGCAGACTAAGCGTATGAGTAACTCTCTGTTTGTGCTCTATTTT |
| | | GGTTTGAATCACCATCATGATCAGCTCGCGCATCACACGGTTTGTTT |
| | | CGGCCCGCGTTACCGCGAACTGATTGACGAGATTTTTAATCATGAT |
| | | GGCCTCGCAGAAGACTTCTCACTTTATCTGCACGCGCCCTGTGTCAC |
| | | GGATTCGTCACTGGCGCCTGAAGGTTGCGGCAGTTACTATGTGTTG |
| | | GCGCCGGTGCCGCATTTAGGCACCGCGAACCTCGACTGGACGGTTG |
| | | AGGGGCCAAAACTACGCGACCGTATTTTTGAGTACCTTGAGCAGCA |
| | | TTACATGCCTGGCTTACGGAGTCAGCTGGTCACGCACCAGATGTTT |
| | | ACGCCGTTTGATTTTCGCGACCAGCTTAATGCCTATCAGGGCTCAGC |
| | | CTTTTCTGTGGAGCCCGTTCTTACCCAGAGCGCCTGGTTTCGGCCGC |
| | | ATAACCGCGATAAAACCATTACTAATCTCTACCTGGTCGGCGCAGG |
| | | CACGCATCCCGGCGCAGGCATTCCTGGCGTCATCGGCTCGGCAAAA |
| | | GCGACAGCAGGTTTGATGCTGGAGGATCTGATTTAA |
| 34 | cipA-trLUT2 | ATGATTAATGACATGCATCCTTCTTTAATTAAAGATAAAGATATAG |
| | | TAGATGACGTAATGCTACGTAGCTGTAAAATCATTGCAATGAAAGT |
| | | TATGCCAGACAAAGTTATGCAAGTTATGGTGACTGTATTAATGCAT |
| | | GATGGCGTATGTGAAGAAATGCTTTTGAAATGGAATCTGCTAGACA |
| | | ATAGAGGCATGGCGATTTATAAAGTTCTGATGGAAGCGCTTTGCGC |
| | | TAAGAAAGATGTGAAAATTAGCACCGTAGGAAAAGTAGGTCCTCTC |
| | | GGCTGCGATTACATTAATTGCGTAGAAATCTCTATGATGGCATCCG |
| | | GCGGTGGCAGCTCTGGCAGCGAATCTTGTGTGGCGGTTCGCGAAGA |
| | | CTTTGCCGATGAAGAAGACTTCGTCAAAGCCGGTGGCTCTGAAATC |
| | | CTGTTTGTGCAGATGCAGCAAAACAAAGACATGGATGAACAAAGT |
| | | AAACTGGTTGATAAACTGCCGCCGATTTCCATCGGTGATGGCGCGC |
| | | TGGACCTGGTTGTGATTGGTTGTGGTCCGGCCGGTCTGGCACTGGC |
| | | AGCTGAAAGCGCCAAACTGGGCCTGAAAGTCGGTCTGATCGGCCCG |
| | | GATCTGCCGTTTACCAACAATTATGGTGTGTGGGAAGATGAATTTA |
| | | ATGACCTGGGCCTGCAAAAATGCATTGAACATGTGTGGCGTGAAAC |
| | | GATCGTTTATCTGGATGACGATAAACCGATTACCATTGGTCGTGCG |
| | | TACGGCCGTGTGAGCCGTCGCCTGCTGCACGAAGAACTGCTGCGTC |
| | | GCTGTGTTGAATCAGGTGTCTCGTATCTGAGTTCCAAAGTTGATAGT |
| | | ATTACGGAAGCATCCGATGGTCTGCGTCTGGTCGCTTGTGACGATA |
| | | ACAATGTGATTCCGTGTCGCCTGGCTACCGTGGCGTCAGGTGCCGC |
| | | CTCGGGTAAACTGCTGCAATATGAAGTTGGTGGCCCGCGTGTCTGT |
| | | GTGCAAACCGCGTATGGTGTTGAAGTCGAAGTGGAAAACTCACCGT |
| | | ACGACCCGGATCAGATGGTTTTTATGGATTATCGTGACTACACCAA |
| | | TGAAAAAGTCCGCAGCCTGGAAGCGGAATATCCGACCTTCCTGTAC |
| | | GCCATGCCGATGACGAAATCACGCCTGTTTTTCGAAGAAACCTGTC |
| | | TGGCCTCGAAAGATGTGATGCCGTTTGACCTGCTGAAAACCAAACT |
| | | GATGCTGCGTCTGGATACGCTGGGCATTCGCATCCTGAAAACCTAT |
| | | GAAGAAGAATGGTCATACATTCCGGTTGGTGGCTCGCTGCCGAACA |
| | | CGGAACAGAAAAATCTGGCATTTGGTGCAGCTGCGAGCATGGTCCA |
| | | TCCGGCTACCGGCTATTCTGTTGTCCGTAGTCTGTCCGAAGCCCCGA |
| | | AATACGCAAGTGTTATTGCTGAAATCCTGCGTGAAGAAACCACGAA |
| | | ACAGATTAACAGCAATATCTCTCGCCAAGCGTGGGATACCCTGTGG |
| | | CCGCCAGAACGTAAACGCCAGCGTGCGTTTTTCCTGTTTGGTCTGGC |
| | | CCTGATTGTGCAATTCGATACGGAAGGCATCCGCAGCTTTTTCCGTA |
| | | CCTTTTTCCGCCTGCCGAAATGGATGTGGCAGGGTTTTCTGGGCAGC |
| | | ACCCTGACGTCTGGCGATCTGGTTCTGTTTGCACTGTATATGTTCGT |
| | | CATTAGCCCGAACAATCTGCGCAAAGGTCTGATTAACCACCTGATC |
| | | TCTGATCCGACCGGCGCTACGATGATCAAAACCTACCTGAAAGTGT |
| | | AA |
| 35 | cipA-trLCYB | ATGATTAATGACATGCATCCTTCTTTAATTAAAGATAAAGATATAG |
| | | TAGATGACGTAATGCTACGTAGCTGTAAAATCATTGCAATGAAAGT |
| | | TATGCCAGACAAAGTTATGCAAGTTATGGTGACTGTATTAATGCAT |
| | | GATGGCGTATGTGAAGAAATGCTTTTGAAATGGAATCTGCTAGACA |
| | | ATAGAGGCATGGCGATTTATAAAGTTCTGATGGAAGCGCTTTGCGC |
| | | TAAGAAAGATGTGAAAATTAGCACCGTAGGAAAAGTAGGTCCTCTC |
| | | GGCTGCGATTACATTAATTGCGTAGAAATCTCTATGATGGTCGTGTC |
| | | GGGGAGCGCGGCCTTGCTGGATTTAGTCCCCGAAACCAAGAAAGA |
| | | GAACCTGGACTTTGAGTTACCCCTTTATGATACGAGTAAATCCCAA |
| | | GTTGTCGATCTGGCAATCGTCGGAGGGGGCCCGGCAGGGTTAGCAG |
| | | TGGCCCAGCAAGTAAGTGAGGCCGGTCTTAGCGTGTGCTCGATTGA |
| | | CCCCAGTCCCAAGTTGATCTGGCCCAACAATTATGGAGTATGGGTC |
| | | GATGAATTTGAAGCTATGGACCTGTTAGACTGCCTGGATACTACTT |
| | | GGAGTGGAGCAGTTGTGTATGTAGATGAAGGAGTTAAAAAAGATC |
| | | TGTCACGTCCTTATGGACGTGTAAACCGCAAACAGTTAAAGTCAAA |
| | | GATGCTGCAAAAATGTATCACGAACGGTGTGAAGTTTCATCAGTCC |
| | | AAAGTGACCAATGTAGTTCACGAGGAGGCAAATTCAACGGTTGTCT |
| | | GTAGCGACGGTGTGAAAATCCAAGCCAGCGTAGTCCTGGATGCGAC |
| | | GGGGTTTTCGCGTTGCCTGGTTCAATACGACAAGCCCTACAACCCA |
| | | GGCTACCAAGTTGCCTACGGAATTGTAGCAGAAGTAGATGGGCACC |
| | | CCTTCGACGTTGACAAAATGGTTTTTATGGACTGGCGTGATAAGCA |
| | | CCTGGATAGTTACCCAGAATTGAAGGAGCGCAATTCAAAAATTCCT |
| | | ACATTCCTTTATGCGATGCCTTTCTCGTCCAATCGCATCTTCTTGGA |
| | | GGAGACCTCCCTGGTAGCTCGCCCGGGTCTTCGCATGGAAGATATC |
| | | CAGGAACGCATGGCCGCTCGCTTAAAGCACTTAGGTATCAACGTGA |
| | | AGCGTATTGAAGAAGATGAACGCTGTGTAATCCCCATGGGTGGGCC |
| | | ACTGCCCGTGTTACCTCAACGTGTGGTAGGTATCGGTGGCACGGCC |
| | | GGCATGGTTCATCCTAGTACCGGTTACATGGTAGCCCGCACACTGG |
| | | CAGCGGCACCTATTGTCGCAAATGCAATCGTTCGTTATTTGGGTTCC |
| | | CCCTCGTCAAACTCCTTACGCGGAGACCAGTTAAGTGCCGAAGTTT |
| | | GGCGCGACCTTTGGCCCATCGAACGTCGTCGCCAACGCGAATTCTT |
| | | CTGTTTTGGGATGGATATCTTATTAAAACTGGACTTAGATGCGACCC |
| | | GCCGCTTCTTCGACGCATTCTTCGATCTTCAGCCACACTACTGGCAC |
| | | GGATTTTTATCATCGCGTTTATTTCTGCCCGAGTTATTGGTATTCGG |
| | | CTTGAGTCTTTTTTCTCATGCGTCGAACACTTCCCGCCTGGAGATCA |
| | | TGACTAAAGGCACCGTACCATTGGCTAAGATGATCAATAACTTAGT |
| | | GCAGGATCGCGACTAA |

2-3. Analysis of Lutein Production

Six plasmids, including the newly constructed plasmids, were combined and introduced into the WLGB-RPP strain, thereby constructing three new lutein-producing strains, LUT2, LUT3, and LUT4 (Table 5).

These strains were cultured under the same conditions as those for LUT1, and as a result, it was confirmed that the largest amount of lutein (0.84 mg l⁻¹) was produced in the LUT4 strain, which possesses pLUT1, pLUT3 and pLUT5 and can bring trLUT2 and trLCYB together (FIG. 1b).

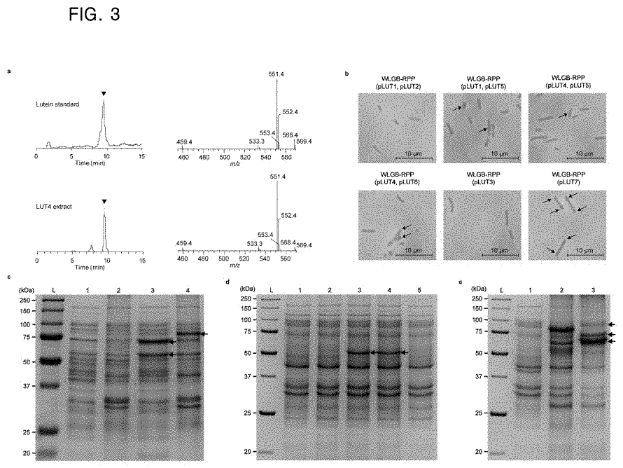

Lutein produced in the LUT4 strain was analyzed by high-performance liquid chromatography-mass spectrometry (HPLC-MS) analysis (FIG. 3a), and intracellular production of PCIs was analyzed by microscopic observation (FIG. 3b) and SDS-PAGE analysis (FIG. 3d). However, it was confirmed that alpha-carotene and beta-carotene were still produced in amounts of 6.74 and 4.50 mg l⁻¹, respectively (FIG. 1b).

TABLE 5
Genes inserted into each strain

| Strain name | Inserted genes |
|---|---|
| LUT2 | crtE, crtB, cipA-crtI, cipA-trLUT2, cipA-trLCYB, |
| | trLUT5, trLUT1, ATR2 |
| LUT3 | crtE, crtB, cipA-crtI, cipA-trLUT2, trLCYB, trLUT5, |
| | trLUT1, ATR2 |
| LUT4 | crtE, crtB, crtI, cipA-trLUT2, cipA-trLCYB, trLUT5, |
| | trLUT1, ATR2 |

### Example 3. Increase in Production Ratio of Alpha-Carotene

It was confirmed in Example 2 that lutein was successfully produced, but a significant amount of beta-carotene still remained in the cells (FIG. 1b). As shown in FIG. 1a, trLCYB is involved in both the reaction that synthesizes beta-carotene from lycopene and the reaction that synthesizes alpha-carotene from delta-carotene. The results in Example 2 suggest that the substrate tunnel composed of trLUT2 and trLCYB is not sufficient to block the metabolic flux to the beta-carotene production pathway.

Thus, this time, a point mutation (G404E) in trLCYB was induced so that trLCYB could produce a higher rate of alpha-carotene (Li, Z. R. et al. Plant Cell 21, 1798-1812, 2009). The trLCYBmut gene was prepared by synthesis, and pLUT5M was constructed by replacing cipA-trLCYB of pLUT5 with cipA-trLCYBmut using the same primers and cloning method.

As a result of flask culture of the LUT4M strain having pLUT1, pLUT3 and pLUT5M, it was confirmed that alpha-carotene and beta-carotene were produced in amounts of 9.11 and 1.43 mg l⁻¹ (FIG. 1b). This means that, due to the G404E mutation induced in trLCYB, the ratio of alpha-carotene production to beta-carotene production increased effectively from 1.50 to 6.37. At this time, alpha-carotene production increased from 6.74 to 9.11 mg l⁻¹, but lutein production was 1.70 mg l⁻¹, which did not increase proportionally to alpha-carotene production. As a result, it could be seen that the two P450 enzymes that convert alpha-carotene into lutein are limiting factors.

Strain Name: LUT4M

Inserted genes: crtE, crtB, crtI, cipA-trLUT2, cipA-trLCYBmut, trLUT5, trLUT1, and ATR2

SEQ ID NO 36: cipA-trLCYBmut gene sequence
ATGATTAATGACATGCATCCTTCTTTAATTAAAGATAAAGATATAGTAGA
TGACGTAATGCTACGTAGCTGTAAAATCATTGCAATGAAAGTTATGCCAG
ACAAAGTTATGCAAGTTATGGTGACTGTATTAATGCATGATGGCGTATGT
GAAGAAATGCTTTTGAAATGGAATCTGCTAGACAATAGAGGCATGGCGAT
TTATAAAGTTCTGATGGAAGCGCTTTGCGCTAAGAAAGATGTGAAAATTA
GCACCGTAGGAAAAGTAGGTCCTCTCGGCTGCGATTACATTAATTGCGTA
GAAATCTCTATGATGGTCGTGTCGGGGAGCGCGGCCTTGCTGGATTTAGT
CCCCGAAACCAAGAAAGAGAACCTGGACTTTGAGTTACCCCTTTATGATA
CGAGTAAATCCCAAGTTGTCGATCTGGCAATCGTCGGAGGGGGCCCGGCA
GGGTTAGCAGTGGCCCAGCAAGTAAGTGAGGCCGGTCTTAGCGTGTGCTC
GATTGACCCCAGTCCCAAGTTGATCTGGCCCAACAATTATGGAGTATGGG
TCGATGAATTTGAAGCTATGGACCTGTTAGACTGCCTGGATACTACTTGG
AGTGGAGCAGTTGTGTATGTAGATGAAGGAGTTAAAAAAGATCTGTCACG
TCCTTATGGACGTGTAAACCGCAAACAGTTAAAGTCAAAGATGCTGCAAA
AATGTATCACGAACGGTGTGAAGTTTCATCAGTCCAAAGTGACCAATGTA
GTTCACGAGGAGGCAAATTCAACGGTTGTCTGTAGCGACGGTGTGAAAAT
CCAAGCCAGCGTAGTCCTGGATGCGACGGGGTTTTCGCGTTGCCTGGTTC
AATACGACAAGCCCTACAACCCAGGCTACCAAGTTGCCTACGGAATTGTA
GCAGAAGTAGATGGGCACCCCTTCGACGTTGACAAAATGGTTTTTATGGA
CTGGCGTGATAAGCACCTGGATAGTTACCCAGAATTGAAGGAGCGCAATT
CAAAAATTCCTACATTCCTTTATGCGATGCCTTTCTCGTCCAATCGCATC
TTCTTGGAGGAGACCTCCCTGGTAGCTCGCCCGGGTCTTCGCATGGAAGA
TATCCAGGAACGCATGGCCGCTCGCTTAAAGCACTTAGGTATCAACGTGA
AGCGTATTGAAGAAGATGAACGCTGTGTAATCCCCATGGGTGGGCCACTG
CCCGTGTTACCTCAACGTGTGGTAGGTATCGGTGGCACGGCCGGCATGGT
TCATCCTAGTACCGGTTACATGGTAGCCCGCACACTGGCAGCGGCACCTA
TTGTCGCAAATGCAATCGTTCGTTATTTGGGTTCCCCCTCGTCAAACTCC
TTACGCGGAGACCAGTTAAGTGCCGAAGTTTGGCGCGACCTTTGGCCCAT
CGAACGTCGTCGCCAACGCGAATTCTTCTGTTTTGGGATGGATATCTTAT
TAAAACTGGACTTAGATGCGACCCGCCGCTTCTTCGACGCATTCTTCGAT
CTTCAGCCACACTACTGGCACGAATTTTTATCATCGCGTTTATTTCTGCC
CGAGTTATTGGTATTCGGCTTGAGTCTTTTTTCTCATGCGTCGAACACTT
CCCGCCTGGAGATCATGACTAAAGGCACCGTACCATTGGCTAAGATGATC
AATAACTTAGTGCAGGATCGCGACTAA
SEQ ID NO 58: trLCYBmut gene sequence
ATGGTCGTGTCGGGGAGCGCGGCCTTGCTGGATTTAGTCCCCGAAACCAA
GAAAGAGAACCTGGACTTTGAGTTACCCCTTTATGATACGAGTAAATCCC
AAGTTGTCGATCTGGCAATCGTCGGAGGGGGCCCGGCAGGGTTAGCAGTG
GCCCAGCAAGTAAGTGAGGCCGGTCTTAGCGTGTGCTCGATTGACCCCAG
TCCCAAGTTGATCTGGCCCAACAATTATGGAGTATGGGTCGATGAATTTG
AAGCTATGGACCTGTTAGACTGCCTGGATACTACTTGGAGTGGAGCAGTT
GTGTATGTAGATGAAGGAGTTAAAAAAGATCTGTCACGTCCTTATGGACG
TGTAAACCGCAAACAGTTAAAGTCAAAGATGCTGCAAAAATGTATCACGA
ACGGTGTGAAGTTTCATCAGTCCAAAGTGACCAATGTAGTTCACGAGGAG
GCAAATTCAACGGTTGTCTGTAGCGACGGTGTGAAAATCCAAGCCAGCGT
AGTCCTGGATGCGACGGGGTTTTCGCGTTGCCTGGTTCAATACGACAAGC
CCTACAACCCAGGCTACCAAGTTGCCTACGGAATTGTAGCAGAAGTAGAT
GGGCACCCCTTCGACGTTGACAAAATGGTTTTTATGGACTGGCGTGATAA
GCACCTGGATAGTTACCCAGAATTGAAGGAGCGCAATTCAAAAATTCCTA
CATTCCTTTATGCGATGCCTTTCTCGTCCAATCGCATCTTCTTGGAGGAG
ACCTCCCTGGTAGCTCGCCCGGGTCTTCGCATGGAAGATATCCAGGAACG
CATGGCCGCTCGCTTAAAGCACTTAGGTATCAACGTGAAGCGTATTGAAG
AAGATGAACGCTGTGTAATCCCCATGGGTGGGCCACTGCCCGTGTTACCT
CAACGTGTGGTAGGTATCGGTGGCACGGCCGGCATGGTTCATCCTAGTAC
CGGTTACATGGTAGCCCGCACACTGGCAGCGGCACCTATTGTCGCAAATG
CAATCGTTCGTTATTTGGGTTCCCCCTCGTCAAACTCCTTACGCGGAGAC
CAGTTAAGTGCCGAAGTTTGGCGCGACCTTTGGCCCATCGAACGTCGTCG
CCAACGCGAATTCTTCTGTTTTGGGATGGATATCTTATTAAAACTGGACT
TAGATGCGACCCGCCGCTTCTTCGACGCATTCTTCGATCTTCAGCCACAC
TACTGGCACGAATTTTTATCATCGCGTTTATTTCTGCCCGAGTTATTGGT
ATTCGGCTTGAGTCTTTTTTCTCATGCGTCGAACACTTCCCGCCTGGAGA
TCATGACTAAAGGCACCGTACCATTGGCTAAGATGATCAATAACTTAGTG
CAGGATCGCGACTAA

### Example 4. Construction of Electron Tunnel

4-1. Selection of Genes for Constructing Electron Tunnel

The eukaryotic P450 system generally consists of P450 and P450 reductases associated with membranes. P450 reductase contains flavin adenine dinucleotide (FAD) and flavin mononucleotide (FMN) factors. Since electrons generated by oxidation of nicotinamide adenine phosphorylated dinucleotide (NADPH) must be transferred to P450 through P450 reductase, the physical distance between the two enzymes is an important factor in electron transfer efficiency.

As a result of SDS-PAGE analysis, it was confirmed that the two P450 enzymes were mostly located in the cytoplasm, whereas ATR2 was located in the cell membrane (FIG. 3c).

Thus, with reference to the study wherein bacterial P450 was assembled with ferredoxin and ferredoxin reductase using proliferative cell nuclear antigen (PCNA) constituting a heterogeneous triad (Haslinger, K. et al., Microb) Cell Fact 19, 2020), the present inventors adopted an approach in which electron transfer occurs through an assembly of trLUT5, trLUT1 and ATR2. It was expected that, by doing so, electrons generated in the NADPH oxidation reaction by ATR2 could be immediately transferred to trLUT5 and trLUT1. In the present invention, the CipB scaffold protein from P. luminescens, which has a smaller molecular size than PCNA and forms stable PCIs separated from CipA, was used as a mediator (FIG. 1c). pLUT7 was constructed by replacing ATR2, trLUT5 and trLUT1 of pLUT3 with cipB-ATR2, cipB-trLUT5 and cipB-trLUT1, respectively.

4-2. Plasmid Construction

A detailed method for construction of pLUT7 is as follows. First, cipB from P. luminescens was synthesized and used. cipB was amplified with each of a combination of cipB_F1/cipB_R1 primers, a combination of cipB_F1/cipB_R2 primers and a combination of cipB_F1/cipB_R3 primers, and then ligated by PCR to ATR2, trLUT5 and trLUT1 amplified with ATR2_F1/R1, trLUT5_F/R1, and trLUT1_F/R1 primers, respectively. The ligated gene fragments were inserted into the EcoRI/BamHI cleavage sites of the pTrc99a plasmid, thereby constructing pTrc-cipB-ATR2, pTrc-cipB-trLUT5, pTrc-cipB-trLUT1 plasmids, respectively.

Thereafter, the cipB-trLUT5 and cipB-trLUT1 gene fragments ligated with the trc promoter were amplified from the pTrc-cipB-trLUT5 and pTrc-cipB-trLUT1 plasmids, respectively, using a combination of pTrc_F1/trLUT5_R2 primers and a combination of pTrc_F2/trLUT1_R2 primers, and then inserted into the BamHI/XbaI and XbaI/SalI cleavage sites of the pTrc-cipB-ATR2 plasmid, respectively.

Table 6 below shows the sequences of the primers used in construction of the plasmids.

TABLE 6

| SEQ ID | | |
|---|---|---|
| NO | Name | Sequence |
| 37 | cipB_F1 | AGACAGGAATTCTTCACACAGGAAAC |
| | | AATGATTATCAAGAAAGATATTCTGC |
| 38 | cipB_R1 | GAAGAAGAAGAAGAAGACATGATTTC |
| | | CACACCCACAAT |
| 39 | cipB_R2 | ACTTGAGCTGCTGAACATGATTTCCA |
| | | CACCCACAAT |
| 40 | cipB_R3 | GGTTTTTCGATGGAACTCATGATTTC |
| | | CACACCCACAAT |

TABLE 7
Gene sequences for constructing electron
tunnel

| SEQ ID NO | Name | Sequence |
|---|---|---|
| 41 | cipB-ATR2 | ATGATTATCAAGAAAGATATTCTGCTTAACGAAGAACTTATTGT |
| | | TGATGACGACTTGAAGGTGGGTAAAGTAGAGAAAGTCAATATT |
| | | GATATTTTGTCTCCCTCATCGGTTATTGTGTCTTTGAACATCCTT |
| | | GGTGTGGTGGATGACTTCCATCTGCTTTTGGTAGATGATAAGGA |
| | | TAAAGACAAAATCGTATTGTTGTATCTGTCTCTGCTTCGCGTCC |
| | | TTCACGAGAAACTGGACGTCAAGGTTAAGGTCGCGAAATCCAA |
| | | ACTTACAAAGATTAAATACATTGTGGGTGTGGAAATCATGTCTT |
| | | CTTCTTCTTCTTCTTCTACCTCTATGATCGACCTGATGGCTGCTA |
| | | TCATCAAAGGTGAACCGGTTATCGTTTCTGACCCGGCTAACGCT |
| | | TCTGCTTACGAATCTGTTGCTGCTGAACTGTCTTCTATGTTAATT |
| | | GAGAATCGTCAGTTTGCTATGATCGTTACAACATCCATCGCGGT |
| | | CCTTATTGGTTGTATCGTTATGTTGGTCTGGCGCCGCTCTGGTTC |
| | | CGGTAACTCTAAACGTGTGGAACCGCTTAAACCGCTGGTGATC |
| | | AAACCTCGTGAGGAGGAAATCGACGATGGACGTAAAAAAGTAA |
| | | CAATCTTTTTCGGAACGCAGACTGGCACTGCGGAAGGTTTTGCC |
| | | AAGGCATTAGGTGAGGAAGCTAAAGCTCGTTATGAAAAGACGC |
| | | GCTTCAAGATTGTTGATCTGGACGATTACGCTGCAGACGATGAT |
| | | GAATACGAGGAAAAATTAAAAAAAGAGGATGTAGCTTTCTTCT |
| | | TCTTAGCTACGTATGGCGATGGTGAACCGACAGATAATGCCGCT |
| | | CGTTTTTATAAGTGGTTTACCGAAGGCAATGATCGTGGTGAGTG |
| | | GTTGAAAAACTTAAAATATGGGGTTTTCGGGCTGGGCAATCGTC |
| | | AATACGAGCACTTTAACAAGGTCGCGAAAGTGGTCGATGACAT |
| | | TCTGGTTGAGCAAGGCGCACAGCGTCTGGTACAAGTAGGGTTA |
| | | GGGGATGATGACCAGTGTATCGAAGATGATTTCACAGCTTGGC |
| | | GCGAAGCATTGTGGCCCGAGTTGGATACGATTCTGCGCGAAGA |
| | | GGGCGATACGGCTGTTGCCACACCCTACACAGCCGCAGTATTA |
| | | GAGTATCGCGTAAGCATCCATGATAGCGAGGATGCCAAATTCA |
| | | ATGATATTAACCTTGCTAACGGAAACGGGTATACAGTTTTTGAC |
| | | GCTCAACATCCGTATAAGGCCAACGTTGCGGTCAAACGTGAAT |
| | | TGCACACCCCGGAGTCCGACCGTTCCTGTATCCATCTGGAATTT |
| | | GATATTGCGGGATCAGGTTTAACATACGAAACTGGAGATCACG |
| | | TTGGTGTTCTGTGCGATAACTTATCCGAGACGGTGGATGAGGCA |
| | | CTGCGCCTTTTAGACATGTCCCCTGACACGTATTTTAGCTTGCAT |
| | | GCTGAAAAAGAGGACGGTACTCCGATCAGTAGCTCGCTGCCAC |
| | | CGCCGTTTCCACCGTGCAATTTACGCACGGCTTTAACACGTTAC |
| | | GCGTGCCTGTTGTCATCTCCTAAGAAATCCGCCTTAGTGGCTTT |
| | | GGCTGCACACGCTAGTGATCCCACTGAGGCCGAGCGCTTGAAA |
| | | CACTTAGCAAGCCCTGCAGGTAAAGACGAGTACTCCAAGTGGG |
| | | TAGTAGAGTCACAGCGTAGTTTATTGGAGGTGATGGCCGAGTTT |
| | | CCTAGTGCGAAGCCACCGTTGGGAGTTTTCTTTGCCGGGGTGGC |
| | | TCCGCGTTTGCAACCACGTTTTTATAGCATCAGTAGTTCTCCAA |
| | | AAATCGCCGAGACTCGCATTCACGTTACATGTGCCCTGGTCTAC |
| | | GAAAAAATGCCGACTGGGCGCATCCACAAGGGTGTATGCTCGA |
| | | CTTGGATGAAGAACGCCGTACCCTACGAAAAGTCTGAAAACTG |
| | | CAGCTCGGCGCCAATCTTCGTACGCCAGTCCAATTTCAAGTTGC |
| | | CGTCAGATTCAAAGGTACCGATCATTATGATCGGTCCAGGAAC |
| | | GGGGTTAGCTCCGTTCCGTGGGTTCTTACAGGAACGCTTAGCAC |
| | | TGGTCGAGTCGGGGGTAGAATTGGGCCCCTCCGTCTTGTTTTTC |
| | | GGGTGTCGTAACCGTCGCATGGACTTCATCTATGAAGAAGAGC |
| | | TGCAACGTTTCGTGGAAAGTGGGGCGCTTGCTGAACTGTCGGTG |
| | | GCGTTTTCCCGCGAAGGACCCACGAAAGAATATGTTCAACACA |
| | | AAATGATGGACAAAGCGTCGGATATCTGGAACATGATTTCACA |
| | | GGGCGCTTATTTATATGTATGTGGCGATGCGAAAGGCATGGCG |
| | | CGTGACGTCCACCGTTCTCTGCACACCATTGCGCAAGAGCAAG |
| | | GTAGCATGGATTCAACGAAAGCAGAAGGCTTCGTGAAGAATTT |
| | | ACAAACCTCTGGGCGCTATCTTCGTGATGTGTGGTAA |
| 42 | cipB-trLUT5 | ATGATTATCAAGAAAGATATTCTGCTTAACGAAGAACTTATTGT |
| | | TGATGACGACTTGAAGGTGGGTAAAGTAGAGAAAGTCAATATT |
| | | GATATTTTGTCTCCCTCATCGGTTATTGTGTCTTTGAACATCCTT |
| | | GGTGTGGTGGATGACTTCCATCTGCTTTTGGTAGATGATAAGGA |
| | | TAAAGACAAAATCGTATTGTTGTATCTGTCTCTGCTTCGCGTCC |
| | | TTCACGAGAAACTGGACGTCAAGGTTAAGGTCGCGAAATCCAA |
| | | ACTTACAAAGATTAAATACATTGTGGGTGTGGAAATCATGTTCA |
| | | GCAGCTCAAGTAACGGCCGCGATCCACTTGAGGAAAACTCAGT |
| | | ACCGAATGGCGTGAAAAGCCTGGAGAAATTGCAGGAGGAAAA |
| | | ACGCCGTGCTGAATTGTCGGCCCGTATCGCATCCGGTGCATTCA |
| | | CCGTTCGTAAGAGCAGCTTCCCCAGCACTGTCAAGAATGGTCTT |
| | | AGCAAAATCGGGATTCCTAGCAACGTATTGGATTTTATGTTCGA |
| | | CTGGACAGGAAGTGACCAGGACTATCCAAAAGTACCTGAGGCG |
| | | AAGGGGTCAATTCAAGCTGTTCGCAACGAGGCGTTTTTTATTCC |
| | | GCTTTACGAACTTTTTTTAACTTATGGTGGGATCTTTCGCCTGAC |
| | | ATTTGGCCCTAAAAGCTTCCTTATTGTTAGCGATCCCTCAATCG |
| | | CCAAACATATCCTGAAGGATAACGCCAAAGCTTACTCCAAGGG |
| | | CATTTTAGCGGAAATCTTAGACTTCGTGATGGGTAAAGGTCTGA |
| | | TCCCGGCCGATGGCGAGATTTGGCGTCGCCGTCGCCGTGCTATC |
| | | GTGCCGGCTCTTCATCAGAAGTATGTCGCTGCAATGATCAGCCT |
| | | TTTCGGTGAAGCCTCAGATCGTTTGTGTCAGAAGTTAGACGCGG |
| | | CAGCCTTGAAAGGGGAGGAGGTCGAAATGGAGAGCCTGTTCTC |
| | | ACGTTTAACTTTGGACATTATTGGTAAAGCCGTATTTAATTATG |
| | | ACTTTGACTCCCTTACCAACGACACGGGGGTGATTGAGGCTGTC |
| | | TACACGGTGCTGCGCGAAGCAGAAGACCGTTCTGTTAGCCCTAT |
| | | CCCGGTTTGGGATATCCCTATTTGGAAAGACATCTCTCCGCGTC |
| | | AGCGTAAAGTTGCAACAAGCTTGAAATTAATTAACGACACACT |
| | | TGATGACTTAATCGCAACATGTAAGCGTATGGTTGAGGAAGAA |
| | | GAGTTACAGTTTCATGAGGAATATATGAACGAACGTGATCCGT |
| | | CCATTTTGCATTTTTTACTTGCTAGTGGTGACGACGTCAGCTCCA |
| | | AACAGTTACGTGACGACCTGATGACTATGCTGATCGCTGGTCAT |
| | | GAAACATCAGCAGCTGTGTTGACCTGGACATTTTACTTGCTTAC |
| | | AACGGAGCCGTCAGTGGTGGCGAAGCTTCAGGAGGAGGTGGAC |
| | | TCCGTTATTGGAGACCGCTTCCCTACAATTCAAGACATGAAGAA |
| | | ATTAAAATATACAACTCGCGTTATGAACGAATCTCTGCGCTTGT |
| | | ATCCCCAACCACCTGTTTTAATTCGTCGCTCTATTGACAATGAC |
| | | ATCTTAGGCGAATACCCTATCAAGCGTGGTGAGGACATCTTCAT |
| | | TTCTGTGTGGAATTTGCATCGCAGCCCCTTGCACTGGGATGATG |
| | | CGGAAAAATTTAATCCCGAGCGTTGGCCACTGGATGGTCCCAA |
| | | CCCAAATGAGACGAATCAGAATTTCAGCTATTTGCCTTTTGGCG |
| | | GAGGACCGCGTAAGTGTATTGGAGATATGTTCGCCAGCTTTGA |
| | | GAATGTGGTAGCCATCGCCATGCTGATCCGTCGTTTTAACTTTC |
| | | AAATTGCACCAGGAGCGCCCCCCGTAAAGATGACCACCGGTGC |
| | | TACTATTCATACGACCGAAGGCTTGAAATTAACAGTAACGAAA |
| | | CGTACAAAACCATTAGATATTCCGTCCGTTCCGATCCTTCCTAT |
| | | GGACACTTCGCGCGATGAAGTTAGCTCGGCCCTTTCTTAA |
| 43 | cipB-trLUT1 | ATGATTATCAAGAAAGATATTCTGCTTAACGAAGAACTTATTGT |
| | | TGATGACGACTTGAAGGTGGGTAAAGTAGAGAAAGTCAATATT |
| | | GATATTTTGTCTCCCTCATCGGTTATTGTGTCTTTGAACATCCTT |
| | | GGTGTGGTGGATGACTTCCATCTGCTTTTGGTAGATGATAAGGA |
| | | TAAAGACAAAATCGTATTGTTGTATCTGTCTCTGCTTCGCGTCC |
| | | TTCACGAGAAACTGGACGTCAAGGTTAAGGTCGCGAAATCCAA |
| | | ACTTACAAAGATTAAATACATTGTGGGTGTGGAAATCATGAGTT |
| | | CCATCGAAAAACCGAAACCGAAACTGGAAACCAACTCATCGAA |
| | | AAGTCAGTCCTGGGTGTCTCCGGATTGGCTGACCACCCTGACCC |
| | | GTACCCTGAGCAGCGGCAAAAACGACGAATCCGGTATTCCGAT |
| | | CGCAAATGCTAAACTGGATGATGTTGCGGATCTGCTGGGCGGC |
| | | GCGCTGTTTCTGCCGCTGTATAAATGGATGAACGAATATGGTCC |
| | | GATTTACCGCCTGGCGGCCGGTCCGCGTAATTTTGTCATTGTGA |
| | | GCGATCCGGCGATCGCCAAACATGTGCTGCGTAACTATCCGAA |
| | | ATACGCCAAAGGCCTGGTGGCAGAAGTTAGCGAATTTCTGTTC |
| | | GGCTCTGGTTTTGCAATTGCTGAAGGTCCGCTGTGGACCGCCCG |
| | | TCGCCGTGCAGTGGTTCCGAGTCTGCACCGCCGTTACCTGTCCG |
| | | TTATCGTCGAACGCGTGTTCTGCAAATGTGCGGAACGTCTGGTT |
| | | GAAAAACTGCAACCGTATGCTGAAGATGGCAGTGCGGTGAATA |
| | | TGGAAGCCAAATTTTCCCAAATGACCCTGGACGTTATCGGTCTG |
| | | TCACTGTTCAACTACAACTTCGATTCACTGACCACGGACTCGCC |
| | | GGTCATCGAAGCAGTGTACACCGCTCTGAAAGAAGCGGAACTG |
| | | CGCTCTACGGATCTGCTGCCGTATTGGAAAATTGACGCACTGTG |
| | | CAAAATCGTTCCGCGCCAGGTCAAAGCAGAAAAAGCTGTCACC |
| | | CTGATTCGTGAAACGGTGGAAGACCTGATTGCGAAATGTAAAG |
| | | AAATCGTGGAACGCGAAGGCGAACGTATTAACGATGAAGAATA |
| | | CGTTAATGATGCGGACCCGAGCATCCTGCGCTTCCTGCTGGCCT |
| | | CTCGTGAAGAAGTCAGTTCCGTGCAGCTGCGCGATGACCTGCTG |
| | | TCTATGCTGGTCGCCGGCCATGAAACCACGGGTTCAGTGCTGAC |
| | | CTGGACGCTGTATCTGCTGTCGAAAAACTCATCGGCCCTGCGTA |
| | | AAGCACAAGAAGAAGTTGATCGCGTCCTGGAAGGTCGTAACCC |
| | | GGCCTTCGAAGACATTAAAGAACTGAAATACATCACCCGCTGC |
| | | ATCAATGAAAGTATGCGTCTGTACCCGCATCCGCCGGTTCTGAT |
| | | TCGCCGTGCACAGGTCCCGGATATTCTGCCGGGCAACTATAAA |
| | | GTGAATACCGGTCAAGACATTATGATCTCCGTTTACAATATCCA |
| | | CCGCAGCTCTGAAGTCTGGGAAAAAGCAGAAGAATTTCTGCCG |
| | | GAACGTTTCGATATTGACGGCGCTATCCCGAACGAAACCAACA |
| | | CGGATTTCAAATTCATCCCGTTTAGCGGCGGTCCGCGCAAATGT |
| | | GTGGGTGATCAGTTCGCTCTGATGGAAGCGATCGTGGCGCTGG |
| | | CCGTGTTTCTGCAACGTCTGAACGTGGAACTGGTTCCGGATCAA |
| | | ACCATTAGCATGACCACGGGCGCCACGATCCACACCACGAATG |
| | | GTCTGTATATGAAAGTTTCTCAACGTTAA |

4-3. Analysis of Lutein Production

As a result of flask culture of the LUT5M strain having pLUT1, pLUT5M and pLUT7, it was confirmed that lutein was produced in the LUT5M strain in an amount of (5.80 mg l⁻¹), which was 3.41 times more than that produced in the LUT4M strain (FIG. 1d). The intracellular production of PCIs composed of trLUT5, trLUT1 and ATR2 was analyzed by microscopic observation and SDS-PAGE analysis (FIG. 3e). As a result, it was confirmed that alpha-carotene and beta-carotene were produced together in amounts of 8.23 and 1.34 mg l⁻¹, respectively.

Strain name: LUT5M

Inserted genes: crtE, crtB, crtI, cipA-trLUT2, cipA-trLCYBmut, cipB-trLUT5, cipB-trLUT1, and cipB-ATR2

### Example 5. Enhancement of C5 Heme Production Pathway

5-1. Selection of Genes for Enhancing C5 Heme Production Pathway

In order to further increase lutein production, the activities of trLUT5 and trLUT1 to convert residual alpha-carotene into lutein should be increased. Two P450 enzymes use heme, which serves as a mediator to transfer electrons from NADPH to oxygen, as a cofactor. If the intracellular heme concentration is low, the function of P450 may be reduced (FIG. 2a). Thus, to enhance the metabolic flux of the C5 heme biosynthesis pathway (Zhao, X. R., et al., Nat Catal 1, 720-728, 2018), E. coli hemA^{fbr}, hemL, hemB and hemH genes (which encode feedback-resistant glutamyl-tRNA reductase, glutamate-1-semialdehyde 2,1-aminomutase, porphobilinogen synthase, and ferrochelatase, respectively) were overexpressed.

pHEM2, which expresses hemAfbr and hemL, and pHEM2, which expresses hemA^{fbr}, hemL, hemB and hemH genes, were constructed and then introduced into LUT5M, thereby constructing LUT5MH1 and LUT5MH2 strains, respectively.

5-2. Plasmid Construction

A detailed plasmid construction method is as follows. The hemA^{fbr}, hemL, hemB and hemH genes were all amplified from the genome of E. coli W3110. First, to construct pHEM2, hemA^{fbr} and hemL genes were amplified using hemA_F1/R primers and hemL_F/R1 primers, respectively, and then inserted into the NcoI/BamHI and BamHI/PstI cleavage sites of the pTrcCDF plasmid, respectively.

Before construction of pHEM2, the pTrcCDF plasmid linearized with pTrcCDF_F/R, hemB amplified with hemB_F/R, and hemH amplified with hemH_F/R1 were assembled together by Gibson's method, thereby constructing a pTrc-hemBH plasmid. A hemBH operon ligated with the trc promoter was amplified from pTrc-hemBH using a combination of pTrc_F3/hemH_R2 primers, and then inserted into the SphI cleavage site of pHEM1, thereby constructing pHEM2.

Table 8 below shows the sequences of the plasmids used in plasmid construction.

TABLE 8

| SEQ | | |
|---|---|---|
| ID | | |
| NO | Name | Sequence |
| 44 | hemA_F1 | AGACAGCCATGGGTACCAAGAAGCTTTTAGCA |
| | | CTCGGTATCAAC |
| 45 | hemA_R | AGACAGGGATCCCTACTCCAGCCCGAGGCT |
| 46 | hemL_F | AGACAGGGATCCTTTCACACAGGAAACAGACC |
| | | ATGAGTAAGTCTGAAAATCTTTAC |
| 47 | hemL_R1 | AGACAGCTGCAGTCACAACTTCGCAAACAC |
| 48 | pTrcCDF_F | TCTAGAGTCGACCTGCAG |
| 49 | pTrcCDF_R | TCTGTTTCCTGTGTGAAATT |
| 50 | hemB_F | AACAATTTCACACAGGAAACAATGACAGACTT |
| | | AATCCAACG |
| 51 | hemB_R | TTAACGCAGAATCTTCTTCTC |
| 52 | hemH_F | CTGAGAAGAAGATTCTGCGTTAATTCACACAG |
| | | GAAACAATGCGTCAGACTAAAACC |
| 53 | hemH_R1 | CTGCAGGTCGACTCTAGATTAGCGATACGCGG |
| | | CAAC |
| 54 | pTrc_F3 | AGACAGGCATGCTTGACAATTAATCATCCGGC |
| 55 | hemH_R2 | AGACAGGCATGCTTAGCGATACGCGGCAAC |

5-3. Analysis of Lutein Production

As a result of flask culture, it was confirmed that the LUT5MH1 and LUT5MH2 strains produced lutein in amounts of 10.34 and 6.04 mg l⁻¹, respectively (FIG. 2b). This means that the increase in heme production had a positive effect on the increase in the synthesis of lutein from alpha-carotene catalyzed by the two P450 enzymes.

TABLE 9
Genes inserted into each strain

| Strain name | Inserted genes |
|---|---|
| LUT5MH1 | crtE, crtB, crtI, cipA-trLUT2, cipA-trLCYBmut, |
| | cipB-trLUT5, cipB-trLUT1, cipB-ATR2, hemA^{fbr}, |
| | hemL |
| LUT5MH2 | crtE, crtB, crtI, cipA-trLUT2, cipA-trLCYBmut, |
| | cipB-trLUT5, cipB-trLUT1, cipB-ATR2, |
| | hem^{fbr}, hemL, hemB, hemH |

SEQ ID NO 56: hemA^{fbr} gene
ATGGGTACCAAGAAGCTTTTAGCACTCGGTATCAACCATAAAACGGCACC
TGTATCGCTGCGAGAACGTGTATCGTTTTCGCCGGATAAGCTCGATCAGG
CGCTTGACAGCCTGCTTGCGCAGCCGATGGTGCAGGGCGGCGTGGTGCTG
TCGACGTGCAACCGCACGGAACTTTATCTTAGCGTTGAAGAGCAGGACAA
CCTGCAAGAGGCGTTAATCCGCTGGCTTTGCGATTATCACAATCTTAATG
AAGAAGATCTGCGTAAAAGCCTCTACTGGCATCAGGATAACGACGCGGTT
AGCCATTTAATGCGTGTTGCCAGCGGCCTGGATTCACTGGTTCTGGGGGA
GCCGCAGATCCTCGGTCAGGTTAAAAAAGCGTTTGCCGATTCGCAAAAAG
GTCATATGAAGGCCAGCGAACTGGAACGCATGTTCCAGAAATCTTTCTCT
GTCGCGAAACGCGTTCGCACTGAAACAGATATCGGTGCCAGCGCTGTGTC
TGTCGCTTTTGCGGCTTGTACGCTGGCGCGGCAGATCTTTGAATCGCTCT
CTACGGTCACAGTGTTGCTGGTAGGCGCGGGCGAAACTATCGAGCTGGTG
GCGCGTCATCTGCGCGAACACAAAGTACAGAAGATGATTATCGCCAACCG
CACTCGCGAACGTGCCCAAATTCTGGCAGATGAAGTCGGCGCGGAAGTGA
TTGCCCTGAGTGATATCGACGAACGTCTGCGCGAAGCCGATATCATCATC
AGTTCCACCGCCAGCCCGTTACCGATTATCGGGAAAGGCATGGTGGAGCG
CGCATTAAAAAGCCGTCGCAACCAACCAATGCTGTTGGTGGATATTGCCG
TTCCGCGCGATGTTGAGCCGGAAGTTGGCAAACTGGCGAATGCTTATCTT
TATAGCGTTGATGATCTGCAAAGCATCATTTCGCACAACCTGGCGCAGCG
TAAAGCCGCAGCGGTTGAGGCGGAAACTATTGTCGCTCAGGAAACCAGCG
AATTTATGGCGTGGCTGCGAGCACAAAGCGCCAGCGAAACCATTCGCGAG
TATCGCAGCCAGGCAGAGCAAGTTCGCGATGAGTTAACCGCCAAAGCGTT
AGCGGCCCTTGAGCAGGGCGGCGACGCGCAAGCCATTATGCAGGATCTGG
CATGGAAACTGACTAACCGCTTGATCCATGCGCCAACGAAATCACTTCAA
CAGGCCGCCCGTGACGGGGATAACGAACGCCTGAATATTCTGCGCGACAG
CCTCGGGCTGGAGTAG
SEQ ID NO 57: hemL gene
ATGAGTAAGTCTGAAAATCTTTACAGCGCAGCGCGCGAGCTGATCCCTGG
CGGTGTGAACTCCCCTGTTCGCGCCTTTACTGGCGTGGGCGGCACTCCAC
TGTTTATCGAAAAAGCGGACGGCGCTTATCTGTACGATGTTGATGGCAAA
GCCTATATCGATTATGTCGGTTCCTGGGGGCCGATGGTGCTGGGCCATAA
CCATCCGGCAATCCGCAATGCCGTGATTGAAGCCGCCGAGCGTGGTTTAA
GCTTTGGTGCACCAACCGAAATGGAAGTGAAAATGGCGCAACTGGTGACC
GAACTGGTCCCGACCATGGATATGGTGCGCATGGTGAACTCCGGCACTGA
AGCGACCATGAGCGCCATCCGCCTGGCCCGTGGTTTTACCGGTCGCGACA
AAATTATTAAATTTGAAGGGTGTTACCATGGTCACGCTGACTGCCTGCTG
GTGAAAGCCGGTTCTGGCGCACTCACGTTAGGCCAGCCAAACTCGCCGGG
CGTTCCGGCAGATTTCGCCAAATATACCTTAACCTGTACTTATAATGATC
TGGCTTCTGTACGCGCCGCATTTGAGCAATACCCGCAAGAGATTGCCTGT
ATTATCGTCGAGCCGGTGGCAGGCAATATGAACTGTGTTCCGCCGCTGCC
AGAGTTCCTGCCAGGTCTGCGCGCGCTGTGCGACGAATTTGGCGCGTTGC
TGATCATCGATGAAGTGATGACCGGTTTCCGCGTAGCGCTAGCTGGCGCA
CAGGATTATTACGGCGTAGTGCCAGATTTAACCTGCCTCGGCAAAATCAT
CGGCGGTGGAATGCCGGTAGGCGCATTCGGTGGTCGTCGTGATGTAATGG
ATGCGCTGGCCCCGACGGGTCCGGTCTATCAGGCGGGTACGCTTTCCGGT
AACCCGATTGCGATGGCAGCGGGTTTCGCCTGTCTGAATGAAGTCGCGCA
GCCGGGCGTTCACGAAACGCTGGATGAGCTGACAACACGTCTGGCAGAAG
GTCTGCTGGAAGCGGCAGAAGAAGCCGGAATTCCGCTGGTCGTTAACCAC
GTTGGCGGCATGTTCGGTATTTTCTTTACCGACGCCGAGTCCGTGACGTG
CTATCAGGATGTGATGGCCTGTGACGTGGAACGCTTTAAGCGTTTCTTCC
ATATGATGCTGGACGAAGGTGTTTACCTGGCACCGTCAGCGTTTGAAGCG
GGCTTTATGTCCGTGGCGCACAGCATGGAAGATATCAATAACACCATCGA
TGCTGCACGTCGGGTGTTTGCGAAGTTGTGA

### Example 6. Culture Condition Optimization

Next, culture conditions having effects on the production of exogenous enzymes including P450 and the growth of cells were optimized.

Culture media (MR and R/2), culture temperatures (22, 25, 28 and 30° C.), and IPTG concentrations (0, 0.01, 0.02, 0.05, 0.1, 0.2, 0.5 and 1 mM) for inducing gene expression were tested under various conditions. As a result, it was confirmed that lutein production was higher when R/2 medium was used than when MR medium was used (FIG. 2c). Thus, the remaining culture conditions were tested using R/2 medium.

R/2 medium (pH 6.8) contains, per liter, 2 g (NH₄)₂HPO₄, 6.75 g KH₂PO₄, 0.85 g citric acid, 0.7 g MgSO₄.7H₂O, 5 ml trace metal solution (TMS) [containing, per liter of 5 M HCl solution, 10 g FeSO₄.7H₂O, 2.25 g ZnSO₄.7H₂O, 1 g CuSO₄.5H₂O, 0.5 g MnSO₄.5H₂O, 0.23 g Na₂B₄O₇.10H₂O, 2 g CaCl₂.2H₂O, and 0.1 g (NH₄)₆Mo₇O₂₄], and MR medium (pH 6.8) contains, per liter, 4 g (NH₄)₂HPO₄, 6.67 g KH₂PO₄, 0.8 g citric acid, 0.8 g MgSO₄.7H₂O, and 5 ml TMS.

Each of the media was further supplemented with 20 g l⁻¹ of glycerol, 3 g l⁻¹ of yeast extract, 50 mg l⁻¹ of kanamycin (Km) , 34 mg l⁻¹ of chloramphenicol (Cm) , 100 mg l⁻¹ of ampicillin (Ap), and 50 mg l⁻¹ of spectinomycin (Spc).

As a result, it was confirmed that, when the culture temperature was lowered to 28° C., lutein production increased to 16.29 mg l⁻¹ (FIG. 2d), and when the IPTG concentration was reduced to 0.05 mM, lutein production further increased to 23.50 mg l⁻¹ (FIG. 2e). Under these conditions, it was confirmed that alpha-carotene production and beta-carotene production decreased to 3.04 and 0.66 mg l⁻¹, respectively.

Next, fed-batch culture of the LUT5MH1 strain was performed in R/2 medium. After the initially supplied carbon source was completely exhausted, a feed solution containing 800 g l⁻¹ of glycerol was supplied using a pH-stat nutrient supply strategy. With reference to the flask culture results described above, the culture temperature was maintained at 28° C. from seed culture. When the optical density at 600 nm (OD600) reached 20 to 30, IPTG was added to a final concentration of 0.05 mM.

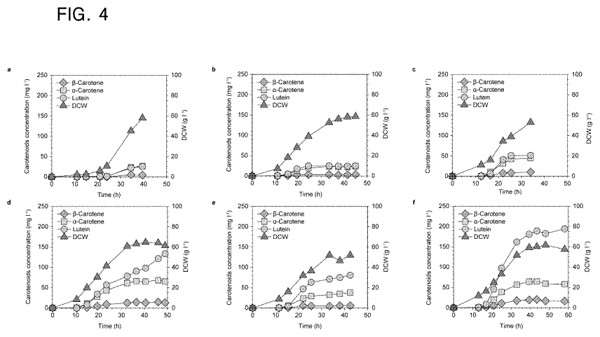

It was confirmed that, when fed-batch culture was performed under these conditions, 25.47 mg l⁻¹ (0.44 mg gDCW⁻¹) of lutein was produced with a productivity of 0.64 mg l⁻¹ h⁻¹ (FIG. 4a). This value was similar to the concentration of lutein produced by flask culture. Optimal culture conditions at the flask level may differ from the optimal culture conditions for fed-batch culture having different conditions including the final cell density level.

Thus, optimization of culture conditions at the fed-batch level was performed. First, the induction phase was shortened by increasing the culture temperature before addition of IPTG from 28° C. to 37° C. (FIG. 4b). Considering the high cell density, the IPTG concentration was reduced to 0.2 mM (FIG. 4c), 0.5 mM (FIGS. 4d), and 1 mM (FIG. 4e), and then lutein production was compared.

As a result, it was confirmed that, when fed batch culture was performed under the conditions where the initial cell culture temperature was increased to 37° C. and the culture temperature was reduced to 28° C. when the OD₆₀₀ reached 23.4, followed by addition of 0.5 mM of IPTG, the lutein production concentration and productivity increased the most to 133.44 mg l⁻¹ (2.17 mg gDCW⁻¹) and 2.72 mg l⁻¹ h⁻¹, respectively (FIG. 4d). FIG. 2f shows photographs of the incubator during fed-batch culture of the LUT5MH1 strain.

### Example 7. Increase in Lutein Production through Carbon Starvation During Fed-Batch Culture

It was confirmed that, when the supply solution was not added for 2 hours after the depletion of the initially supplied carbon source during the fed-batch culture, lutein production and productivity further increased to 194.20 mg l⁻¹ (3.38 mg gDCW⁻¹) and 3.35 mg l⁻¹ h⁻¹, respectively (FIG. 4f).

To confirm the consistency of these results, fed-batch culture was performed again under the same conditions, and as a result, it was confirmed that lutein production and productivity further increased to 218.0 mg l⁻¹ (4.01 mg gDCW⁻¹) and 5.01 mg l⁻¹ h⁻¹, respectively (FIG. 2g). In order to find out the factors causing the increase in lutein production, the transcriptomes of cells cultured under carbon-starved conditions and cells cultured under existing conditions were analyzed and compared. As a result it was confirmed that the change in the expression levels of stress response genes increased the final concentration of lutein by slowing the oxidation rate of intracellular carotenoids.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

Using the highly efficient lutein-producing recombinant microbial strain according to the present invention, it is possible to replace an existing lutein production method that relies on labor-intensive and inefficient plant extraction and to produce lutein in a more environmentally friendly and sustainable way. In addition, the strain development strategy used in the present invention is useful because it may be used to construct a recombinant strain for the efficient production of useful compounds with complex metabolic pathways and to establish an efficient production method, and it may be applied throughout the gradually expanding biochemical market.

1. A recombinant microorganism having ability to produce lutein, which is obtained by introducing a lutein biosynthetic pathway into a host cell having ability to produce farnesyl diphosphate (FPP).

2. The recombinant microorganism of claim 1, wherein the host cell having ability to produce farnesyl diphosphate (FPP) is a host cell which lacks at least one gene selected from the group consisting of lacI (lactose operon repressor) gene, gdhA (NADP-specific glutamate dehydrogenase) gene and gpmB (phosphoglycerate mutase) gene and in which at least one gene selected from the group consisting of dxs (1-deoxyxylulose-5-phosphate synthase) gene, idi (isopentenyl diphosphate (IPDP) isomerase) gene, ispA (geranyltranstransferase/dimethylallyltranstransferase) gene and pps (PEP synthase) gene has been introduced or amplified.

3. The recombinant microorganism of claim 1, wherein the lutein biosynthetic pathway is a lutein biosynthetic pathway into which at least one gene selected from the group consisting of crtE (geranylgeranyl pyrophosphate synthase) gene, crtB (phytoene synthase) gene, crtI (phytoene dehydrogenase) gene, LUT2 (lycopene ε-cyclase) gene, LCYB (lycopene β-cyclase) gene, ATR2 (cytochrome P450 reductase) gene, LUT5 (β-carotene 3-hydroxylase) gene and LUT1 (carotene ε-monooxygenase) gene has been introduced.

4. The recombinant microorganism of claim 3, wherein the LCYB gene encodes a G451E mutant protein.

5. The recombinant microorganism of claim 3, wherein any one or more selected from the group consisting of substrate tunnel formation, electron tunnel formation, and C5 heme production pathway modification has been introduced so that the ability to produce lutein is further enhanced.

6. The recombinant microorganism of claim 5, wherein the substrate tunnel formation is performed by introducing cipA gene.

7. The recombinant microorganism of claim 6, wherein the introducing the cipA gene comprises modifying any one or more genes, selected from the group consisting of crtl, LUT2 and LCYB, into any one or more genes selected from the group consisting of cipA-crtl, cipA-LUT2 and cipA-LCYB.

8. The recombinant microorganism of claim 5, wherein the electron tunnel formation is performed by introducing cipB gene.

9. The recombinant microorganism of claim 8, wherein the introducing the cipB gene comprises modifying any one or more genes, selected from the group consisting of ATR2, LUT5 and LUT1, into any one or more genes selected from the group consisting of cipB-ATR2, cipB-LUT5 and cipB-LUT1.

10. The recombinant microorganism of claim 5, wherein the C5 heme production pathway modification is performed by introducing any one or more genes selected from the group consisting of hemA, hemL, hemB and hemH.

11. The recombinant microorganism of claim 10, wherein the hemA gene encodes a mutant protein resistant to feedback inhibition.

12. The recombinant microorganism of claim 1, wherein the host cell is selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhodobacter, Zymomonas, Bacillus, Staphylococcus, Lactococcus, Streptococcus, Lactobacillus, Clostridium, Corynebacterium, Streptomyces, Bifidobacterium, Cyanobacterium, and Cyclobacterium.

13. A recombinant microorganism having enhanced ability to produce lutein, which is obtained by introducing a lutein biosynthetic pathway into a host cell having ability to produce farnesyl diphosphate (FPP) and introducing any one or more selected from the group consisting of substrate tunnel formation, electron tunnel formation, and C5 heme production pathway modification.

14. The recombinant microorganism of claim 13, wherein the lutein biosynthetic pathway is a lutein biosynthetic pathway into which at least one gene selected from the group consisting of crtE (geranylgeranyl pyrophosphate synthase) gene, crtB (phytoene synthase) gene, crtI (phytoene dehydrogenase) gene, LUT2 (lycopene ε-cyclase) gene, LCYB (lycopene (β-cyclase) gene, ATR2 (cytochrome P450 reductase) gene, LUT5 (β-carotene 3-hydroxylase) gene and LUT1 (carotene ε-monooxygenase) gene has been introduced.

15. The recombinant microorganism of claim 13, wherein the substrate tunnel formation is performed by introducing cipA gene.

16. The recombinant microorganism of claim 13, wherein the electron tunnel formation is performed by introducing cipB gene.

17. The recombinant microorganism of claim 13, wherein the C5 heme production pathway modification is performed by introducing or amplifying any one or more genes selected from the group consisting of hemA, hemL, hemB and hemH.

18. The recombinant microorganism of claim 13, wherein the host cell is selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhodobacter, Zymomonas, Bacillus, Staphylococcus, Lactococcus, Streptococcus, Lactobacillus, Clostridium, Corynebacterium, Streptomyces, Bifidobacterium, Cyanobacterium, and Cyclobacterium.

19. A method for producing lutein comprising steps of: (a) producing lutein by culturing the recombinant microorganism of claim 1; and (b) recovering the produced lutein.

20. The method of claim 19, wherein step (a) comprises a step of making the temperature of the culturing after expression of lutein different from the temperature of the culturing before the expression, and inducing carbon starvation after the expression.


SEQUENCE LISTING (58 sequences)

SEQ ID NO: 1 - DNA, 47 bp, Artificial Sequence
  Synthetic Construct
agacaggagctcttcacacaggaaacaatgtatccgtttataaggac

SEQ ID NO: 2 - DNA, 20 bp, Artificial Sequence
  Synthetic Construct
ttaactgacggcagcgagtt

SEQ ID NO: 3 - DNA, 55 bp, Artificial Sequence
  Synthetic Construct
aactcgctgccgtcagttaattcacacaggaaacaatgaaaccaactacggtaat

SEQ ID NO: 4 - DNA, 32 bp, Artificial Sequence
  Synthetic Construct
agacagtctagattagagcgggcgctgccaga

SEQ ID NO: 5 - DNA, 42 bp, Artificial Sequence
  Synthetic Construct
agacaggagctcttcacacaggaaacaatggcatccggcggt

SEQ ID NO: 6 - DNA, 34 bp, Artificial Sequence
  Synthetic Construct
agacagtctagattacactttcaggtaggttttg

SEQ ID NO: 7 - DNA, 42 bp, Artificial Sequence
  Synthetic Construct
agacagtctagattcacacaggaaacaatggtcgtgtcgggg

SEQ ID NO: 8 - DNA, 32 bp, Artificial Sequence
  Synthetic Construct
agacagctgcagttagtcgcgatcctgcacta

SEQ ID NO: 9 - DNA, 47 bp, Artificial Sequence
  Synthetic Construct
agacaggaattcttcacacaggaaacaatgtcttcttcttcttcttc

SEQ ID NO: 10 - DNA, 32 bp, Artificial Sequence
  Synthetic Construct
agacagggatccttaccacacatcacgaagat

SEQ ID NO: 11 - DNA, 44 bp, Artificial Sequence
  Synthetic Construct
agacaggaattcttcacacaggaaacaatgttcagcagctcaag

SEQ ID NO: 12 - DNA, 32 bp, Artificial Sequence
  Synthetic Construct
agacagggatccttaagaaagggccgagctaa

SEQ ID NO: 13 - DNA, 47 bp, Artificial Sequence
  Synthetic Construct
agacaggaattcttcacacaggaaacaatgagttccatcgaaaaacc

SEQ ID NO: 14 - DNA, 32 bp, Artificial Sequence
  Synthetic Construct
agacagggatccttaacgttgagaaactttca

SEQ ID NO: 15 - DNA, 31 bp, Artificial Sequence
  Synthetic Construct
agacagggatccttgacaattaatcatccgg

SEQ ID NO: 16 - DNA, 28 bp, Artificial Sequence
  Synthetic Construct
agacagtctagattaagaaagggccgag

SEQ ID NO: 17 - DNA, 31 bp, Artificial Sequence
  Synthetic Construct
agacagtctagattgacaattaatcatccgg

SEQ ID NO: 18 - DNA, 32 bp, Artificial Sequence
  Synthetic Construct
agacaggtcgacttaacgttgagaaactttca

SEQ ID NO: 19 - DNA, 1443 bp, Artificial Sequence
  Synthetic Construct
atggcatccggcggtggcagctctggcagcgaatcttgtgtggcggttcgcgaagactttgccgatgaagaagacttcgtcaaagccggtggctctgaaatcctgtttgtgcagatgcagcaaaacaaagacatggatgaacaaagtaaactggttgataaactgccgccgatttccatcggtgatggcgcgctggacctggttgtgattggttgtggtccggccggtctggcactggcagctgaaagcgccaaactgggcctgaaagtcggtctgatcggcccggatctgccgtttaccaacaattatggtgtgtgggaagatgaatttaatgacctgggcctgcaaaaatgcattgaacatgtgtggcgtgaaacgatcgtttatctggatgacgataaaccgattaccattggtcgtgcgtacggccgtgtgagccgtcgcctgctgcacgaagaactgctgcgtcgctgtgttgaatcaggtgtctcgtatctgagttccaaagttgatagtattacggaagcatccgatggtctgcgtctggtcgcttgtgacgataacaatgtgattccgtgtcgcctggctaccgtggcgtcaggtgccgcctcgggtaaactgctgcaatatgaagttggtggcccgcgtgtctgtgtgcaaaccgcgtatggtgttgaagtcgaagtggaaaactcaccgtacgacccggatcagatggtttttatggattatcgtgactacaccaatgaaaaagtccgcagcctggaagcggaatatccgaccttcctgtacgccatgccgatgacgaaatcacgcctgtttttcgaagaaacctgtctggcctcgaaagatgtgatgccgtttgacctgctgaaaaccaaactgatgctgcgtctggatacgctgggcattcgcatcctgaaaacctatgaagaagaatggtcatacattccggttggtggctcgctgccgaacacggaacagaaaaatctggcatttggtgcagctgcgagcatggtccatccggctaccggctattctgttgtccgtagtctgtccgaagccccgaaatacgcaagtgttattgctgaaatcctgcgtgaagaaaccacgaaacagattaacagcaatatctctcgccaagcgtgggataccctgtggccgccagaacgtaaacgccagcgtgcgtttttcctgtttggtctggccctgattgtgcaattcgatacggaaggcatccgcagctttttccgtacctttttccgcctgccgaaatggatgtggcagggttttctgggcagcaccctgacgtctggcgatctggttctgtttgcactgtatatgttcgtcattagcccgaacaatctgcgcaaaggtctgattaaccacctgatctctgatccgaccggcgctacgatgatcaaaacctacctgaaagtgtaa

SEQ ID NO: 20 - DNA, 1365 bp, Artificial Sequence
  Synthetic Construct
atggtcgtgtcggggagcgcggccttgctggatttagtccccgaaaccaagaaagagaacctggactttgagttacccctttatgatacgagtaaatcccaagttgtcgatctggcaatcgtcggagggggcccggcagggttagcagtggcccagcaagtaagtgaggccggtcttagcgtgtgctcgattgaccccagtcccaagttgatctggcccaacaattatggagtatgggtcgatgaatttgaagctatggacctgttagactgcctggatactacttggagtggagcagttgtgtatgtagatgaaggagttaaaaaagatctgtcacgtccttatggacgtgtaaaccgcaaacagttaaagtcaaagatgctgcaaaaatgtatcacgaacggtgtgaagtttcatcagtccaaagtgaccaatgtagttcacgaggaggcaaattcaacggttgtctgtagcgacggtgtgaaaatccaagccagcgtagtcctggatgcgacggggttttcgcgttgcctggttcaatacgacaagccctacaacccaggctaccaagttgcctacggaattgtagcagaagtagatgggcaccccttcgacgttgacaaaatggtttttatggactggcgtgataagcacctggatagttacccagaattgaaggagcgcaattcaaaaattcctacattcctttatgcgatgcctttctcgtccaatcgcatcttcttggaggagacctccctggtagctcgcccgggtcttcgcatggaagatatccaggaacgcatggccgctcgcttaaagcacttaggtatcaacgtgaagcgtattgaagaagatgaacgctgtgtaatccccatgggtgggccactgcccgtgttacctcaacgtgtggtaggtatcggtggcacggccggcatggttcatcctagtaccggttacatggtagcccgcacactggcagcggcacctattgtcgcaaatgcaatcgttcgttatttgggttccccctcgtcaaactccttacgcggagaccagttaagtgccgaagtttggcgcgacctttggcccatcgaacgtcgtcgccaacgcgaattcttctgttttgggatggatatcttattaaaactggacttagatgcgacccgccgcttcttcgacgcattcttcgatcttcagccacactactggcacggatttttatcatcgcgtttatttctgcccgagttattggtattcggcttgagtcttttttctcatgcgtcgaacacttcccgcctggagatcatgactaaaggcaccgtaccattggctaagatgatcaataacttagtgcaggatcgcgactaa

SEQ ID NO: 21 - DNA, 1707 bp, Artificial Sequence
  Synthetic Construct
atgttcagcagctcaagtaacggccgcgatccacttgaggaaaactcagtaccgaatggcgtgaaaagcctggagaaattgcaggaggaaaaacgccgtgctgaattgtcggcccgtatcgcatccggtgcattcaccgttcgtaagagcagcttccccagcactgtcaagaatggtcttagcaaaatcgggattcctagcaacgtattggattttatgttcgactggacaggaagtgaccaggactatccaaaagtacctgaggcgaaggggtcaattcaagctgttcgcaacgaggcgttttttattccgctttacgaactttttttaacttatggtgggatctttcgcctgacatttggccctaaaagcttccttattgttagcgatccctcaatcgccaaacatatcctgaaggataacgccaaagcttactccaagggcattttagcggaaatcttagacttcgtgatgggtaaaggtctgatcccggccgatggcgagatttggcgtcgccgtcgccgtgctatcgtgccggctcttcatcagaagtatgtcgctgcaatgatcagccttttcggtgaagcctcagatcgtttgtgtcagaagttagacgcggcagccttgaaaggggaggaggtcgaaatggagagcctgttctcacgtttaactttggacattattggtaaagccgtatttaattatgactttgactcccttaccaacgacacgggggtgattgaggctgtctacacggtgctgcgcgaagcagaagaccgttctgttagccctatcccggtttgggatatccctatttggaaagacatctctccgcgtcagcgtaaagttgcaacaagcttgaaattaattaacgacacacttgatgacttaatcgcaacatgtaagcgtatggttgaggaagaagagttacagtttcatgaggaatatatgaacgaacgtgatccgtccattttgcattttttacttgctagtggtgacgacgtcagctccaaacagttacgtgacgacctgatgactatgctgatcgctggtcatgaaacatcagcagctgtgttgacctggacattttacttgcttacaacggagccgtcagtggtggcgaagcttcaggaggaggtggactccgttattggagaccgcttccctacaattcaagacatgaagaaattaaaatatacaactcgcgttatgaacgaatctctgcgcttgtatccccaaccacctgttttaattcgtcgctctattgacaatgacatcttaggcgaataccctatcaagcgtggtgaggacatcttcatttctgtgtggaatttgcatcgcagccccttgcactgggatgatgcggaaaaatttaatcccgagcgttggccactggatggtcccaacccaaatgagacgaatcagaatttcagctatttgccttttggcggaggaccgcgtaagtgtattggagatatgttcgccagctttgagaatgtggtagccatcgccatgctgatccgtcgttttaactttcaaattgcaccaggagcgccccccgtaaagatgaccaccggtgctactattcatacgaccgaaggcttgaaattaacagtaacgaaacgtacaaaaccattagatattccgtccgttccgatccttcctatggacacttcgcgcgatgaagttagctcggccctttcttaa

SEQ ID NO: 22 - DNA, 1515 bp, Artificial Sequence
  Synthetic Construct
atgagttccatcgaaaaaccgaaaccgaaactggaaaccaactcatcgaaaagtcagtcctgggtgtctccggattggctgaccaccctgacccgtaccctgagcagcggcaaaaacgacgaatccggtattccgatcgcaaatgctaaactggatgatgttgcggatctgctgggcggcgcgctgtttctgccgctgtataaatggatgaacgaatatggtccgatttaccgcctggcggccggtccgcgtaattttgtcattgtgagcgatccggcgatcgccaaacatgtgctgcgtaactatccgaaatacgccaaaggcctggtggcagaagttagcgaatttctgttcggctctggttttgcaattgctgaaggtccgctgtggaccgcccgtcgccgtgcagtggttccgagtctgcaccgccgttacctgtccgttatcgtcgaacgcgtgttctgcaaatgtgcggaacgtctggttgaaaaactgcaaccgtatgctgaagatggcagtgcggtgaatatggaagccaaattttcccaaatgaccctggacgttatcggtctgtcactgttcaactacaacttcgattcactgaccacggactcgccggtcatcgaagcagtgtacaccgctctgaaagaagcggaactgcgctctacggatctgctgccgtattggaaaattgacgcactgtgcaaaatcgttccgcgccaggtcaaagcagaaaaagctgtcaccctgattcgtgaaacggtggaagacctgattgcgaaatgtaaagaaatcgtggaacgcgaaggcgaacgtattaacgatgaagaatacgttaatgatgcggacccgagcatcctgcgcttcctgctggcctctcgtgaagaagtcagttccgtgcagctgcgcgatgacctgctgtctatgctggtcgccggccatgaaaccacgggttcagtgctgacctggacgctgtatctgctgtcgaaaaactcatcggccctgcgtaaagcacaagaagaagttgatcgcgtcctggaaggtcgtaacccggccttcgaagacattaaagaactgaaatacatcacccgctgcatcaatgaaagtatgcgtctgtacccgcatccgccggttctgattcgccgtgcacaggtcccggatattctgccgggcaactataaagtgaataccggtcaagacattatgatctccgtttacaatatccaccgcagctctgaagtctgggaaaaagcagaagaatttctgccggaacgtttcgatattgacggcgctatcccgaacgaaaccaacacggatttcaaattcatcccgtttagcggcggtccgcgcaaatgtgtgggtgatcagttcgctctgatggaagcgatcgtggcgctggccgtgtttctgcaacgtctgaacgtggaactggttccggatcaaaccattagcatgaccacgggcgccacgatccacaccacgaatggtctgtatatgaaagtttctcaacgttaa

SEQ ID NO: 23 - DNA, 57 bp, Artificial Sequence
  Synthetic Construct
aactcgctgccgtcagttaattcacacaggaaacaatgattaatgacatgcatcctt

SEQ ID NO: 24 - DNA, 44 bp, Artificial Sequence
  Synthetic Construct
caattaccgtagttggtttcatcatagagatttctacgcaatta

SEQ ID NO: 25 - DNA, 20 bp, Artificial Sequence
  Synthetic Construct
atgaaaccaactacggtaat

SEQ ID NO: 26 - DNA, 22 bp, Artificial Sequence
  Synthetic Construct
taaatcagatcctccagcatca

SEQ ID NO: 27 - DNA, 60 bp, Artificial Sequence
  Synthetic Construct
tgatgctggaggatctgatttaattcacacaggaaacaatgaataatccgtcgttactca

SEQ ID NO: 28 - DNA, 20 bp, Artificial Sequence
  Synthetic Construct
ttaactgacggcagcgagtt

SEQ ID NO: 29 - DNA, 47 bp, Artificial Sequence
  Synthetic Construct
agacaggagctcttcacacaggaaacaatgattaatgacatgcatcc

SEQ ID NO: 30 - DNA, 43 bp, Artificial Sequence
  Synthetic Construct
gctgccaccgccggatgccatcatagagatttctacgcaatta

SEQ ID NO: 31 - DNA, 47 bp, Artificial Sequence
  Synthetic Construct
agacagtctagattcacacaggaaacaatgattaatgacatgcatcc

SEQ ID NO: 32 - DNA, 40 bp, Artificial Sequence
  Synthetic Construct
gctccccgacacgaccatcatagagatttctacgcaatta

SEQ ID NO: 33 - DNA, 1791 bp, Artificial Sequence
  Synthetic Construct
atgattaatgacatgcatccttctttaattaaagataaagatatagtagatgacgtaatgctacgtagctgtaaaatcattgcaatgaaagttatgccagacaaagttatgcaagttatggtgactgtattaatgcatgatggcgtatgtgaagaaatgcttttgaaatggaatctgctagacaatagaggcatggcgatttataaagttctgatggaagcgctttgcgctaagaaagatgtgaaaattagcaccgtaggaaaagtaggtcctctcggctgcgattacattaattgcgtagaaatctctatgatgaaaccaactacggtaattggtgcaggcttcggtggcctggcactggcaattcgtctacaggctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggaacgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgtatgagtaactctctgtttgtgctctattttggtttgaatcaccatcatgatcagctcgcgcatcacacggtttgtttcggcccgcgttaccgcgaactgattgacgagatttttaatcatgatggcctcgcagaagacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgagtaccttgagcagcattacatgcctggcttacggagtcagctggtcacgcaccagatgtttacgccgtttgattttcgcgaccagcttaatgcctatcagggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttaa

SEQ ID NO: 34 - DNA, 1755 bp, Artificial Sequence
  Synthetic Construct
atgattaatgacatgcatccttctttaattaaagataaagatatagtagatgacgtaatgctacgtagctgtaaaatcattgcaatgaaagttatgccagacaaagttatgcaagttatggtgactgtattaatgcatgatggcgtatgtgaagaaatgcttttgaaatggaatctgctagacaatagaggcatggcgatttataaagttctgatggaagcgctttgcgctaagaaagatgtgaaaattagcaccgtaggaaaagtaggtcctctcggctgcgattacattaattgcgtagaaatctctatgatggcatccggcggtggcagctctggcagcgaatcttgtgtggcggttcgcgaagactttgccgatgaagaagacttcgtcaaagccggtggctctgaaatcctgtttgtgcagatgcagcaaaacaaagacatggatgaacaaagtaaactggttgataaactgccgccgatttccatcggtgatggcgcgctggacctggttgtgattggttgtggtccggccggtctggcactggcagctgaaagcgccaaactgggcctgaaagtcggtctgatcggcccggatctgccgtttaccaacaattatggtgtgtgggaagatgaatttaatgacctgggcctgcaaaaatgcattgaacatgtgtggcgtgaaacgatcgtttatctggatgacgataaaccgattaccattggtcgtgcgtacggccgtgtgagccgtcgcctgctgcacgaagaactgctgcgtcgctgtgttgaatcaggtgtctcgtatctgagttccaaagttgatagtattacggaagcatccgatggtctgcgtctggtcgcttgtgacgataacaatgtgattccgtgtcgcctggctaccgtggcgtcaggtgccgcctcgggtaaactgctgcaatatgaagttggtggcccgcgtgtctgtgtgcaaaccgcgtatggtgttgaagtcgaagtggaaaactcaccgtacgacccggatcagatggtttttatggattatcgtgactacaccaatgaaaaagtccgcagcctggaagcggaatatccgaccttcctgtacgccatgccgatgacgaaatcacgcctgtttttcgaagaaacctgtctggcctcgaaagatgtgatgccgtttgacctgctgaaaaccaaactgatgctgcgtctggatacgctgggcattcgcatcctgaaaacctatgaagaagaatggtcatacattccggttggtggctcgctgccgaacacggaacagaaaaatctggcatttggtgcagctgcgagcatggtccatccggctaccggctattctgttgtccgtagtctgtccgaagccccgaaatacgcaagtgttattgctgaaatcctgcgtgaagaaaccacgaaacagattaacagcaatatctctcgccaagcgtgggataccctgtggccgccagaacgtaaacgccagcgtgcgtttttcctgtttggtctggccctgattgtgcaattcgatacggaaggcatccgcagctttttccgtacctttttccgcctgccgaaatggatgtggcagggttttctgggcagcaccctgacgtctggcgatctggttctgtttgcactgtatatgttcgtcattagcccgaacaatctgcgcaaaggtctgattaaccacctgatctctgatccgaccggcgctacgatgatcaaaacctacctgaaagtgtaa

SEQ ID NO: 35 - DNA, 1677 bp, Artificial Sequence
  Synthetic Construct
atgattaatgacatgcatccttctttaattaaagataaagatatagtagatgacgtaatgctacgtagctgtaaaatcattgcaatgaaagttatgccagacaaagttatgcaagttatggtgactgtattaatgcatgatggcgtatgtgaagaaatgcttttgaaatggaatctgctagacaatagaggcatggcgatttataaagttctgatggaagcgctttgcgctaagaaagatgtgaaaattagcaccgtaggaaaagtaggtcctctcggctgcgattacattaattgcgtagaaatctctatgatggtcgtgtcggggagcgcggccttgctggatttagtccccgaaaccaagaaagagaacctggactttgagttacccctttatgatacgagtaaatcccaagttgtcgatctggcaatcgtcggagggggcccggcagggttagcagtggcccagcaagtaagtgaggccggtcttagcgtgtgctcgattgaccccagtcccaagttgatctggcccaacaattatggagtatgggtcgatgaatttgaagctatggacctgttagactgcctggatactacttggagtggagcagttgtgtatgtagatgaaggagttaaaaaagatctgtcacgtccttatggacgtgtaaaccgcaaacagttaaagtcaaagatgctgcaaaaatgtatcacgaacggtgtgaagtttcatcagtccaaagtgaccaatgtagttcacgaggaggcaaattcaacggttgtctgtagcgacggtgtgaaaatccaagccagcgtagtcctggatgcgacggggttttcgcgttgcctggttcaatacgacaagccctacaacccaggctaccaagttgcctacggaattgtagcagaagtagatgggcaccccttcgacgttgacaaaatggtttttatggactggcgtgataagcacctggatagttacccagaattgaaggagcgcaattcaaaaattcctacattcctttatgcgatgcctttctcgtccaatcgcatcttcttggaggagacctccctggtagctcgcccgggtcttcgcatggaagatatccaggaacgcatggccgctcgcttaaagcacttaggtatcaacgtgaagcgtattgaagaagatgaacgctgtgtaatccccatgggtgggccactgcccgtgttacctcaacgtgtggtaggtatcggtggcacggccggcatggttcatcctagtaccggttacatggtagcccgcacactggcagcggcacctattgtcgcaaatgcaatcgttcgttatttgggttccccctcgtcaaactccttacgcggagaccagttaagtgccgaagtttggcgcgacctttggcccatcgaacgtcgtcgccaacgcgaattcttctgttttgggatggatatcttattaaaactggacttagatgcgacccgccgcttcttcgacgcattcttcgatcttcagccacactactggcacggatttttatcatcgcgtttatttctgcccgagttattggtattcggcttgagtcttttttctcatgcgtcgaacacttcccgcctggagatcatgactaaaggcaccgtaccattggctaagatgatcaataacttagtgcaggatcgcgactaa

SEQ ID NO: 36 - DNA, 1677 bp, Artificial Sequence
  Synthetic Construct
atgattaatgacatgcatccttctttaattaaagataaagatatagtagatgacgtaatgctacgtagctgtaaaatcattgcaatgaaagttatgccagacaaagttatgcaagttatggtgactgtattaatgcatgatggcgtatgtgaagaaatgcttttgaaatggaatctgctagacaatagaggcatggcgatttataaagttctgatggaagcgctttgcgctaagaaagatgtgaaaattagcaccgtaggaaaagtaggtcctctcggctgcgattacattaattgcgtagaaatctctatgatggtcgtgtcggggagcgcggccttgctggatttagtccccgaaaccaagaaagagaacctggactttgagttacccctttatgatacgagtaaatcccaagttgtcgatctggcaatcgtcggagggggcccggcagggttagcagtggcccagcaagtaagtgaggccggtcttagcgtgtgctcgattgaccccagtcccaagttgatctggcccaacaattatggagtatgggtcgatgaatttgaagctatggacctgttagactgcctggatactacttggagtggagcagttgtgtatgtagatgaaggagttaaaaaagatctgtcacgtccttatggacgtgtaaaccgcaaacagttaaagtcaaagatgctgcaaaaatgtatcacgaacggtgtgaagtttcatcagtccaaagtgaccaatgtagttcacgaggaggcaaattcaacggttgtctgtagcgacggtgtgaaaatccaagccagcgtagtcctggatgcgacggggttttcgcgttgcctggttcaatacgacaagccctacaacccaggctaccaagttgcctacggaattgtagcagaagtagatgggcaccccttcgacgttgacaaaatggtttttatggactggcgtgataagcacctggatagttacccagaattgaaggagcgcaattcaaaaattcctacattcctttatgcgatgcctttctcgtccaatcgcatcttcttggaggagacctccctggtagctcgcccgggtcttcgcatggaagatatccaggaacgcatggccgctcgcttaaagcacttaggtatcaacgtgaagcgtattgaagaagatgaacgctgtgtaatccccatgggtgggccactgcccgtgttacctcaacgtgtggtaggtatcggtggcacggccggcatggttcatcctagtaccggttacatggtagcccgcacactggcagcggcacctattgtcgcaaatgcaatcgttcgttatttgggttccccctcgtcaaactccttacgcggagaccagttaagtgccgaagtttggcgcgacctttggcccatcgaacgtcgtcgccaacgcgaattcttctgttttgggatggatatcttattaaaactggacttagatgcgacccgccgcttcttcgacgcattcttcgatcttcagccacactactggcacgaatttttatcatcgcgtttatttctgcccgagttattggtattcggcttgagtcttttttctcatgcgtcgaacacttcccgcctggagatcatgactaaaggcaccgtaccattggctaagatgatcaataacttagtgcaggatcgcgactaa

SEQ ID NO: 37 - DNA, 52 bp, Artificial Sequence
  Synthetic Construct
agacaggaattcttcacacaggaaacaatgattatcaagaaagatattctgc

SEQ ID NO: 38 - DNA, 38 bp, Artificial Sequence
  Synthetic Construct
gaagaagaagaagaagacatgatttccacacccacaat

SEQ ID NO: 39 - DNA, 36 bp, Artificial Sequence
  Synthetic Construct
acttgagctgctgaacatgatttccacacccacaat

SEQ ID NO: 40 - DNA, 38 bp, Artificial Sequence
  Synthetic Construct
ggtttttcgatggaactcatgatttccacacccacaat

SEQ ID NO: 41 - DNA, 2436 bp, Artificial Sequence
  Synthetic Construct
atgattatcaagaaagatattctgcttaacgaagaacttattgttgatgacgacttgaaggtgggtaaagtagagaaagtcaatattgatattttgtctccctcatcggttattgtgtctttgaacatccttggtgtggtggatgacttccatctgcttttggtagatgataaggataaagacaaaatcgtattgttgtatctgtctctgcttcgcgtccttcacgagaaactggacgtcaaggttaaggtcgcgaaatccaaacttacaaagattaaatacattgtgggtgtggaaatcatgtcttcttcttcttcttcttctacctctatgatcgacctgatggctgctatcatcaaaggtgaaccggttatcgtttctgacccggctaacgcttctgcttacgaatctgttgctgctgaactgtcttctatgttaattgagaatcgtcagtttgctatgatcgttacaacatccatcgcggtccttattggttgtatcgttatgttggtctggcgccgctctggttccggtaactctaaacgtgtggaaccgcttaaaccgctggtgatcaaacctcgtgaggaggaaatcgacgatggacgtaaaaaagtaacaatctttttcggaacgcagactggcactgcggaaggttttgccaaggcattaggtgaggaagctaaagctcgttatgaaaagacgcgcttcaagattgttgatctggacgattacgctgcagacgatgatgaatacgaggaaaaattaaaaaaagaggatgtagctttcttcttcttagctacgtatggcgatggtgaaccgacagataatgccgctcgtttttataagtggtttaccgaaggcaatgatcgtggtgagtggttgaaaaacttaaaatatggggttttcgggctgggcaatcgtcaatacgagcactttaacaaggtcgcgaaagtggtcgatgacattctggttgagcaaggcgcacagcgtctggtacaagtagggttaggggatgatgaccagtgtatcgaagatgatttcacagcttggcgcgaagcattgtggcccgagttggatacgattctgcgcgaagagggcgatacggctgttgccacaccctacacagccgcagtattagagtatcgcgtaagcatccatgatagcgaggatgccaaattcaatgatattaaccttgctaacggaaacgggtatacagtttttgacgctcaacatccgtataaggccaacgttgcggtcaaacgtgaattgcacaccccggagtccgaccgttcctgtatccatctggaatttgatattgcgggatcaggtttaacatacgaaactggagatcacgttggtgttctgtgcgataacttatccgagacggtggatgaggcactgcgccttttagacatgtcccctgacacgtattttagcttgcatgctgaaaaagaggacggtactccgatcagtagctcgctgccaccgccgtttccaccgtgcaatttacgcacggctttaacacgttacgcgtgcctgttgtcatctcctaagaaatccgccttagtggctttggctgcacacgctagtgatcccactgaggccgagcgcttgaaacacttagcaagccctgcaggtaaagacgagtactccaagtgggtagtagagtcacagcgtagtttattggaggtgatggccgagtttcctagtgcgaagccaccgttgggagttttctttgccggggtggctccgcgtttgcaaccacgtttttatagcatcagtagttctccaaaaatcgccgagactcgcattcacgttacatgtgccctggtctacgaaaaaatgccgactgggcgcatccacaagggtgtatgctcgacttggatgaagaacgccgtaccctacgaaaagtctgaaaactgcagctcggcgccaatcttcgtacgccagtccaatttcaagttgccgtcagattcaaaggtaccgatcattatgatcggtccaggaacggggttagctccgttccgtgggttcttacaggaacgcttagcactggtcgagtcgggggtagaattgggcccctccgtcttgtttttcgggtgtcgtaaccgtcgcatggacttcatctatgaagaagagctgcaacgtttcgtggaaagtggggcgcttgctgaactgtcggtggcgttttcccgcgaaggacccacgaaagaatatgttcaacacaaaatgatggacaaagcgtcggatatctggaacatgatttcacagggcgcttatttatatgtatgtggcgatgcgaaaggcatggcgcgtgacgtccaccgttctctgcacaccattgcgcaagagcaaggtagcatggattcaacgaaagcagaaggcttcgtgaagaatttacaaacctctgggcgctatcttcgtgatgtgtggtaa

SEQ ID NO: 42 - DNA, 2007 bp, Artificial Sequence
  Synthetic Construct
atgattatcaagaaagatattctgcttaacgaagaacttattgttgatgacgacttgaaggtgggtaaagtagagaaagtcaatattgatattttgtctccctcatcggttattgtgtctttgaacatccttggtgtggtggatgacttccatctgcttttggtagatgataaggataaagacaaaatcgtattgttgtatctgtctctgcttcgcgtccttcacgagaaactggacgtcaaggttaaggtcgcgaaatccaaacttacaaagattaaatacattgtgggtgtggaaatcatgttcagcagctcaagtaacggccgcgatccacttgaggaaaactcagtaccgaatggcgtgaaaagcctggagaaattgcaggaggaaaaacgccgtgctgaattgtcggcccgtatcgcatccggtgcattcaccgttcgtaagagcagcttccccagcactgtcaagaatggtcttagcaaaatcgggattcctagcaacgtattggattttatgttcgactggacaggaagtgaccaggactatccaaaagtacctgaggcgaaggggtcaattcaagctgttcgcaacgaggcgttttttattccgctttacgaactttttttaacttatggtgggatctttcgcctgacatttggccctaaaagcttccttattgttagcgatccctcaatcgccaaacatatcctgaaggataacgccaaagcttactccaagggcattttagcggaaatcttagacttcgtgatgggtaaaggtctgatcccggccgatggcgagatttggcgtcgccgtcgccgtgctatcgtgccggctcttcatcagaagtatgtcgctgcaatgatcagccttttcggtgaagcctcagatcgtttgtgtcagaagttagacgcggcagccttgaaaggggaggaggtcgaaatggagagcctgttctcacgtttaactttggacattattggtaaagccgtatttaattatgactttgactcccttaccaacgacacgggggtgattgaggctgtctacacggtgctgcgcgaagcagaagaccgttctgttagccctatcccggtttgggatatccctatttggaaagacatctctccgcgtcagcgtaaagttgcaacaagcttgaaattaattaacgacacacttgatgacttaatcgcaacatgtaagcgtatggttgaggaagaagagttacagtttcatgaggaatatatgaacgaacgtgatccgtccattttgcattttttacttgctagtggtgacgacgtcagctccaaacagttacgtgacgacctgatgactatgctgatcgctggtcatgaaacatcagcagctgtgttgacctggacattttacttgcttacaacggagccgtcagtggtggcgaagcttcaggaggaggtggactccgttattggagaccgcttccctacaattcaagacatgaagaaattaaaatatacaactcgcgttatgaacgaatctctgcgcttgtatccccaaccacctgttttaattcgtcgctctattgacaatgacatcttaggcgaataccctatcaagcgtggtgaggacatcttcatttctgtgtggaatttgcatcgcagccccttgcactgggatgatgcggaaaaatttaatcccgagcgttggccactggatggtcccaacccaaatgagacgaatcagaatttcagctatttgccttttggcggaggaccgcgtaagtgtattggagatatgttcgccagctttgagaatgtggtagccatcgccatgctgatccgtcgttttaactttcaaattgcaccaggagcgccccccgtaaagatgaccaccggtgctactattcatacgaccgaaggcttgaaattaacagtaacgaaacgtacaaaaccattagatattccgtccgttccgatccttcctatggacacttcgcgcgatgaagttagctcggccctttcttaa

SEQ ID NO: 43 - DNA, 1815 bp, Artificial Sequence
  Synthetic Construct
atgattatcaagaaagatattctgcttaacgaagaacttattgttgatgacgacttgaaggtgggtaaagtagagaaagtcaatattgatattttgtctccctcatcggttattgtgtctttgaacatccttggtgtggtggatgacttccatctgcttttggtagatgataaggataaagacaaaatcgtattgttgtatctgtctctgcttcgcgtccttcacgagaaactggacgtcaaggttaaggtcgcgaaatccaaacttacaaagattaaatacattgtgggtgtggaaatcatgagttccatcgaaaaaccgaaaccgaaactggaaaccaactcatcgaaaagtcagtcctgggtgtctccggattggctgaccaccctgacccgtaccctgagcagcggcaaaaacgacgaatccggtattccgatcgcaaatgctaaactggatgatgttgcggatctgctgggcggcgcgctgtttctgccgctgtataaatggatgaacgaatatggtccgatttaccgcctggcggccggtccgcgtaattttgtcattgtgagcgatccggcgatcgccaaacatgtgctgcgtaactatccgaaatacgccaaaggcctggtggcagaagttagcgaatttctgttcggctctggttttgcaattgctgaaggtccgctgtggaccgcccgtcgccgtgcagtggttccgagtctgcaccgccgttacctgtccgttatcgtcgaacgcgtgttctgcaaatgtgcggaacgtctggttgaaaaactgcaaccgtatgctgaagatggcagtgcggtgaatatggaagccaaattttcccaaatgaccctggacgttatcggtctgtcactgttcaactacaacttcgattcactgaccacggactcgccggtcatcgaagcagtgtacaccgctctgaaagaagcggaactgcgctctacggatctgctgccgtattggaaaattgacgcactgtgcaaaatcgttccgcgccaggtcaaagcagaaaaagctgtcaccctgattcgtgaaacggtggaagacctgattgcgaaatgtaaagaaatcgtggaacgcgaaggcgaacgtattaacgatgaagaatacgttaatgatgcggacccgagcatcctgcgcttcctgctggcctctcgtgaagaagtcagttccgtgcagctgcgcgatgacctgctgtctatgctggtcgccggccatgaaaccacgggttcagtgctgacctggacgctgtatctgctgtcgaaaaactcatcggccctgcgtaaagcacaagaagaagttgatcgcgtcctggaaggtcgtaacccggccttcgaagacattaaagaactgaaatacatcacccgctgcatcaatgaaagtatgcgtctgtacccgcatccgccggttctgattcgccgtgcacaggtcccggatattctgccgggcaactataaagtgaataccggtcaagacattatgatctccgtttacaatatccaccgcagctctgaagtctgggaaaaagcagaagaatttctgccggaacgtttcgatattgacggcgctatcccgaacgaaaccaacacggatttcaaattcatcccgtttagcggcggtccgcgcaaatgtgtgggtgatcagttcgctctgatggaagcgatcgtggcgctggccgtgtttctgcaacgtctgaacgtggaactggttccggatcaaaccattagcatgaccacgggcgccacgatccacaccacgaatggtctgtatatgaaagtttctcaacgttaa

SEQ ID NO: 44 - DNA, 44 bp, Artificial Sequence
  Synthetic Construct
agacagccatgggtaccaagaagcttttagcactcggtatcaac

SEQ ID NO: 45 - DNA, 30 bp, Artificial Sequence
  Synthetic Construct
agacagggatccctactccagcccgaggct

SEQ ID NO: 46 - DNA, 56 bp, Artificial Sequence
  Synthetic Construct
agacagggatcctttcacacaggaaacagaccatgagtaagtctgaaaatctttac

SEQ ID NO: 47 - DNA, 30 bp, Artificial Sequence
  Synthetic Construct
agacagctgcagtcacaacttcgcaaacac

SEQ ID NO: 48 - DNA, 18 bp, Artificial Sequence
  Synthetic Construct
tctagagtcgacctgcag

SEQ ID NO: 49 - DNA, 20 bp, Artificial Sequence
  Synthetic Construct
tctgtttcctgtgtgaaatt

SEQ ID NO: 50 - DNA, 41 bp, Artificial Sequence
  Synthetic Construct
aacaatttcacacaggaaacaatgacagacttaatccaacg

SEQ ID NO: 51 - DNA, 21 bp, Artificial Sequence
  Synthetic Construct
ttaacgcagaatcttcttctc

SEQ ID NO: 52 - DNA, 56 bp, Artificial Sequence
  Synthetic Construct
ctgagaagaagattctgcgttaattcacacaggaaacaatgcgtcagactaaaacc

SEQ ID NO: 53 - DNA, 36 bp, Artificial Sequence
  Synthetic Construct
ctgcaggtcgactctagattagcgatacgcggcaac

SEQ ID NO: 54 - DNA, 32 bp, Artificial Sequence
  Synthetic Construct
agacaggcatgcttgacaattaatcatccggc

SEQ ID NO: 55 - DNA, 30 bp, Artificial Sequence
  Synthetic Construct
agacaggcatgcttagcgatacgcggcaac

SEQ ID NO: 56 - DNA, 1266 bp, Artificial Sequence
  Synthetic Construct
atgggtaccaagaagcttttagcactcggtatcaaccataaaacggcacctgtatcgctgcgagaacgtgtatcgttttcgccggataagctcgatcaggcgcttgacagcctgcttgcgcagccgatggtgcagggcggcgtggtgctgtcgacgtgcaaccgcacggaactttatcttagcgttgaagagcaggacaacctgcaagaggcgttaatccgctggctttgcgattatcacaatcttaatgaagaagatctgcgtaaaagcctctactggcatcaggataacgacgcggttagccatttaatgcgtgttgccagcggcctggattcactggttctgggggagccgcagatcctcggtcaggttaaaaaagcgtttgccgattcgcaaaaaggtcatatgaaggccagcgaactggaacgcatgttccagaaatctttctctgtcgcgaaacgcgttcgcactgaaacagatatcggtgccagcgctgtgtctgtcgcttttgcggcttgtacgctggcgcggcagatctttgaatcgctctctacggtcacagtgttgctggtaggcgcgggcgaaactatcgagctggtggcgcgtcatctgcgcgaacacaaagtacagaagatgattatcgccaaccgcactcgcgaacgtgcccaaattctggcagatgaagtcggcgcggaagtgattgccctgagtgatatcgacgaacgtctgcgcgaagccgatatcatcatcagttccaccgccagcccgttaccgattatcgggaaaggcatggtggagcgcgcattaaaaagccgtcgcaaccaaccaatgctgttggtggatattgccgttccgcgcgatgttgagccggaagttggcaaactggcgaatgcttatctttatagcgttgatgatctgcaaagcatcatttcgcacaacctggcgcagcgtaaagccgcagcggttgaggcggaaactattgtcgctcaggaaaccagcgaatttatggcgtggctgcgagcacaaagcgccagcgaaaccattcgcgagtatcgcagccaggcagagcaagttcgcgatgagttaaccgccaaagcgttagcggcccttgagcagggcggcgacgcgcaagccattatgcaggatctggcatggaaactgactaaccgcttgatccatgcgccaacgaaatcacttcaacaggccgcccgtgacggggataacgaacgcctgaatattctgcgcgacagcctcgggctggagtag

SEQ ID NO: 57 - DNA, 1281 bp, Artificial Sequence
  Synthetic Construct
atgagtaagtctgaaaatctttacagcgcagcgcgcgagctgatccctggcggtgtgaactcccctgttcgcgcctttactggcgtgggcggcactccactgtttatcgaaaaagcggacggcgcttatctgtacgatgttgatggcaaagcctatatcgattatgtcggttcctgggggccgatggtgctgggccataaccatccggcaatccgcaatgccgtgattgaagccgccgagcgtggtttaagctttggtgcaccaaccgaaatggaagtgaaaatggcgcaactggtgaccgaactggtcccgaccatggatatggtgcgcatggtgaactccggcactgaagcgaccatgagcgccatccgcctggcccgtggttttaccggtcgcgacaaaattattaaatttgaagggtgttaccatggtcacgctgactgcctgctggtgaaagccggttctggcgcactcacgttaggccagccaaactcgccgggcgttccggcagatttcgccaaatataccttaacctgtacttataatgatctggcttctgtacgcgccgcatttgagcaatacccgcaagagattgcctgtattatcgtcgagccggtggcaggcaatatgaactgtgttccgccgctgccagagttcctgccaggtctgcgcgcgctgtgcgacgaatttggcgcgttgctgatcatcgatgaagtgatgaccggtttccgcgtagcgctagctggcgcacaggattattacggcgtagtgccagatttaacctgcctcggcaaaatcatcggcggtggaatgccggtaggcgcattcggtggtcgtcgtgatgtaatggatgcgctggccccgacgggtccggtctatcaggcgggtacgctttccggtaacccgattgcgatggcagcgggtttcgcctgtctgaatgaagtcgcgcagccgggcgttcacgaaacgctggatgagctgacaacacgtctggcagaaggtctgctggaagcggcagaagaagccggaattccgctggtcgttaaccacgttggcggcatgttcggtattttctttaccgacgccgagtccgtgacgtgctatcaggatgtgatggcctgtgacgtggaacgctttaagcgtttcttccatatgatgctggacgaaggtgtttacctggcaccgtcagcgtttgaagcgggctttatgtccgtggcgcacagcatggaagatatcaataacaccatcgatgctgcacgtcgggtgtttgcgaagttgtga

SEQ ID NO: 58 - DNA, 1365 bp, Artificial Sequence
  Synthetic Construct
atggtcgtgtcggggagcgcggccttgctggatttagtccccgaaaccaagaaagagaacctggactttgagttacccctttatgatacgagtaaatcccaagttgtcgatctggcaatcgtcggagggggcccggcagggttagcagtggcccagcaagtaagtgaggccggtcttagcgtgtgctcgattgaccccagtcccaagttgatctggcccaacaattatggagtatgggtcgatgaatttgaagctatggacctgttagactgcctggatactacttggagtggagcagttgtgtatgtagatgaaggagttaaaaaagatctgtcacgtccttatggacgtgtaaaccgcaaacagttaaagtcaaagatgctgcaaaaatgtatcacgaacggtgtgaagtttcatcagtccaaagtgaccaatgtagttcacgaggaggcaaattcaacggttgtctgtagcgacggtgtgaaaatccaagccagcgtagtcctggatgcgacggggttttcgcgttgcctggttcaatacgacaagccctacaacccaggctaccaagttgcctacggaattgtagcagaagtagatgggcaccccttcgacgttgacaaaatggtttttatggactggcgtgataagcacctggatagttacccagaattgaaggagcgcaattcaaaaattcctacattcctttatgcgatgcctttctcgtccaatcgcatcttcttggaggagacctccctggtagctcgcccgggtcttcgcatggaagatatccaggaacgcatggccgctcgcttaaagcacttaggtatcaacgtgaagcgtattgaagaagatgaacgctgtgtaatccccatgggtgggccactgcccgtgttacctcaacgtgtggtaggtatcggtggcacggccggcatggttcatcctagtaccggttacatggtagcccgcacactggcagcggcacctattgtcgcaaatgcaatcgttcgttatttgggttccccctcgtcaaactccttacgcggagaccagttaagtgccgaagtttggcgcgacctttggcccatcgaacgtcgtcgccaacgcgaattcttctgttttgggatggatatcttattaaaactggacttagatgcgacccgccgcttcttcgacgcattcttcgatcttcagccacactactggcacgaatttttatcatcgcgtttatttctgcccgagttattggtattcggcttgagtcttttttctcatgcgtcgaacacttcccgcctggagatcatgactaaaggcaccgtaccattggctaagatgatcaataacttagtgcaggatcgcgactaa